SEQUENCE LISTING (25 sequences)

SEQ ID NO: 1 - DNA, 267 bp, Homo sapiens
atggccgaggagctggtcttagagaggtgtgatctggagctggagaccaatggccgagaccaccacacggccgacctgtgccgggagaagctggtggtgcgacggggccagcccttctggctgaccctgcactttgagggccgcaactaccaggccagtgtagacagtctcaccttcagtgtcgtgaccggcccagcccctagccaggaggccgggaccaaggcccgttttccactaagagatgctgtggaggagggtgactggaca

SEQ ID NO: 2 - protein, 89 aa, Homo sapiens
MAEELVLERCDLELETNGRDHHTADLCREKLVVRRGQPFWLTLHFEGRNYQASVDSLTFSVVTGPAPSQEAGTKARFPLRDAVEEGDWT

SEQ ID NO: 3 - DNA, 267 bp, Guinea pig
atggcagaggatctgatcctggagagatgtgatttgcagctggaggtcaatggccgcgaccaccgcacggccgacctgtgccgggagaggctggtgttgcggcggggccagcccttctggctgacgctgcactttgagggccgtggctacgaggctggtgtggacactctcaccttcaacgctgtgaccggcccagatcccagtgaggaggccgggactatggcccggttctcactgtccagtgctgtcgaggggggcacctggtca

SEQ ID NO: 4 - protein, 89 aa, Guinea pig
MAEDLILERCDLQLEVNGRDHRTADLCRERLVLRRGQPFWLTLHFEGRGYEAGVDTLTFNAVTGPDPSEEAGTMARFSLSSAVEGGTWS

SEQ ID NO: 5 - DNA, 267 bp, Bovine
atggccgaggagctggtcctggagagatgtgacctggagctggaggccaatggccgtgaccaccacacagctgacctgtgcagggaaaggctggtagtgcggcggggccagcccttctggctgactctgcactttgagggccggaactatgaggccagcgtggacagcctcaccttttgtgctgtgactggcccagaccccagtgaggaggccgggaccaaggccctcttcaggctgtccgatgctacggaggagggggcctgggca

SEQ ID NO: 6 - protein, 89 aa, Bovine
MAEELVLERCDLELEANGRDHHTADLCRERLVVRRGQPFWLTLHFEGRNYEASVDSLTFCAVTGPDPSEEAGTKALFRLSDATEEGAWA

SEQ ID NO: 7 - DNA, 267 bp, Mouse
atggcagaggagctgctcctggagaggtgtgatttggagattcaggccaatggccgtgaccaccacacggccgacctatgccaagagaaactggtgctgcgtcgtggtcagcgcttccggctgactctgtacttcgagggccgtggctacgaggccagcgtggacagcctcacgttcggtgctgtgaccggcccagatcccagtgaagaggcagggaccaaggcccgcttctcactgtctgacaatgtggaggagggatcttggtca

SEQ ID NO: 8 - protein, 89 aa, Mouse
MAEELLLERCDLEIQANGRDHHTADLCQEKLVLRRGQRFRLTLYFEGRGYEASVDSLTFGAVTGPDPSEEAGTKARFSLSDNVEEGSWS

SEQ ID NO: 9 - DNA, 267 bp, Rat
atggcagaggagctgaacctggagaggtgcgacttggagatacaggccaatggccgtgatcaccacacggccgacctgtgccaacagaaactggtgctgcggcgaggccagcgcttccggctgacactgtacttcgagggccgtggctatgaggccagcgtggacagacttacatttggtgccgtgaccggcccagatcccagtgaagaggcagggaccaaggcccgattctcactgtctgacgatgtggaggagggatcctggtca

SEQ ID NO: 10 - protein, 89 aa, Rat
MAEELNLERCDLEIQANGRDHHTADLCQQKLVLRRGQRFRLTLYFEGRGYEASVDRLTFGAVTGPDPSEEAGTKARFSLSDDVEEGSWS

SEQ ID NO: 11 - DNA, 273 bp, Homo sapiens
gtggtagactggatccagcaggacgatgggtctgtgcacaaatccatcaaccgttccctgatcgttgggctgaagatcagcactaagagcgtgggccgagacgagcgggaggatatcacccacacctacaaatacccagaggggtcctcagaggagagggaggccttcacaagggcgaaccacctgaacaaactggccgagaaggaggagacagggatggccatgcggatccgtgtgggccagagcatgaacatgggcagtgactttgacgtc

SEQ ID NO: 12 - protein, 91 aa, Homo sapiens
VVDWIQQDDGSVHKSINRSLIVGLKISTKSVGRDEREDITHTYKYPEGSSEEREAFTRANHLNKLAEKEETGMAMRIRVGQSMNMGSDFDV

SEQ ID NO: 13 - DNA, 282 bp, Guinea pig
gtggtgaactggatccggcagaaagatgggtccctgcgcaagtccatcaaccatttggttgtggggctgaagatcagtactaagagtgtgggccgcgatgagcgagaggacatcacccacacctacaagtacccagagggatctgaagaggagcgggaagcttttgttagggccaaccacctaaataaactggccacaaaggaagaggctcaggaggaaacgggagtggccatgcggatccgtgtgggccagaacatgactatgggcagtgactttgacatc

SEQ ID NO: 14 - protein, 94 aa, Guinea pig
VVNWIRQKDGSLRKSINHLVVGLKISTKSVGRDEREDITHTYKYPEGSEEEREAFVRANHLNKLATKEEAQEETGVAMRIRVGQNMTMGSDFDI

SEQ ID NO: 15 - DNA, 273 bp, Bovine
gtggtggactggatccggcaggacgatgggtctctgcacaaatccatcaaccactccctggtggtggggctgaagatcagcacaaagtgtgtgggcagagatgatcgggaggacatcacccacagctacaagtacccggaggggtccccagaggaaagggaagccttcacaagagccaaccatctgaacaaactggttaacaaagaggagacaggggtggccatgcggatccgtgtgggcgagggcatgaacagaggctgcgacttcgacgtc

SEQ ID NO: 16 - protein, 91 aa, Bovine
VVDWIRQDDGSLHKSINHSLVVGLKISTKCVGRDDREDITHSYKYPEGSPEEREAFTRANHLNKLVNKEETGVAMRIRVGEGMNRGCDFDV

SEQ ID NO: 17 - DNA, 273 bp, Mouse
gtggtggactggatccggcaggaagatgggtctgtgctcaaatccatcaaccgttccttggtcgtggggcagaagatcagcactaagagtgtgggccgtgatgaccgggaggacatcacccatacctacaagtacccagaggggtcacccgaggagagggaagtcttcaccaaggccaaccacctgaacaaactggcagagaaagaggagacaggggtggccatgcgcatccgagtgggggacagtatgagcatgggcaacgacttcgacgtg

SEQ ID NO: 18 - protein, 91 aa, Mouse
VVDWIRQEDGSVLKSINRSLVVGQKISTKSVGRDDREDITHTYKYPEGSPEEREVFTKANHLNKLAEKEETGVAMRIRVGDSMSMGNDFDV

SEQ ID NO: 19 - DNA, 273 bp, Rat
gtggtggactggatccggcagtcagatgggtctgtgctcaaatccatcaacaattccctggtcgtggggcagaaaatcagcactaagagcgtgggccgtgatgaccgggaggacatcacctatacctacaagtacccagaggggtccccagaggaaagggaagtcttcaccagagccaaccacctgaacaaactggcagagaaagaggagacaggggtggccatgcggatccgagtgggggatggtatgagcttgggcaatgactttgacgtg

SEQ ID NO: 20 - protein, 91 aa, Rat
VVDWIRQSDGSVLKSINNSLVVGQKISTKSVGRDDREDITYTYKYPEGSPEEREVFTRANHLNKLAEKEETGVAMRIRVGDGMSLGNDFDV

SEQ ID NO: 21 - protein, 687 aa, Homo sapiens
MAEELVLERCDLELETNGRDHHTADLCREKLVVRRGQPFWLTLHFEGRNYQASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFQDGILDICLILLDVNPKFLKNAGRDCSRRSSPVYVGRVGSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLTLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLVPLHMGLHKLVVNFESDKLKAVKGFRNVIIGPA

SEQ ID NO: 22 - protein, 690 aa, Guinea pig
MAEDLILERCDLQLEVNGRDHRTADLCRERLVLRRGQPFWLTLHFEGRGYEAGVDTLTFNAVTGPDPSEEAGTMARFSLSSAVEGGTWSASAVDQQDSTVSLLLSTPADAPIGLYRLSLEASTGYQGSSFVLGHFILLYNPRCPADAVYMDSDQERQEYVLTQQGFIYQGSAKFINGIPWNFGQFEDGILDICLMLLDTNPKFLKNAGQDCSRRSRPVYVGRVVSAMVNCNDDQGVLQGRWDNNYSDGVSPMSWIGSVDILRRWKDYGCQRVKYGQCWVFAAVACTVLRCLGIPTRVVTNFNSAHDQNSNLLIEYFRNESGEIEGNKSEMIWNFHCWVESWMTRPDLEPGYEGWQALDPTPQEKSEGTYCCGPVPVRAIKEGHLNVKYDAPFVFAEVNADVVNWIRQKDGSLRKSINHLVVGLKISTKSVGRDEREDITHTYKYPEGSEEEREAFVRANHLNKLATKEEAQEETGVAMRIRVGQNMTMGSDFDIFAYITNGTAESHECQLLLCARIVSYNGVLGPVCSTNDLLNLTLDPFSENSIPLHILYEKYGDYLTESNLIKVRGLLIEPAANSYVLAERDIYLENPEIKIRVLGEPKQNRKLIAEVSLKNPLPVPLLGCIFTVEGAGLTKDQKSVEVPDPVEAGEQAKVRVDLLPTEVGLHKLVVNFECDKLKAVKGYRNVIIGPA

SEQ ID NO: 23 - protein, 691 aa, Bovine
MAEELVLERCDLELEANGRDHHTADLCRERLVVRRGQPFWLTLHFEGRNYEASVDSLTFCAVTGPDPSEEAGTKALFRLSDATEEGAWAAVAADQRDSTLSLHLSTPANAPVGHYRLSLEASTGYQGSSFMLGQFTLLFNSWCPADAVYLDSDEERQEYVLTQQGFIYQGSAKFIKNIPWNFGQFEEGILDICLMLLDVNPKFLRNAGRDCSRRSSPVYVGRVVSGMVNCNDDQGVLLGRWDNNYADGISPMSWIGSVDILRRWKRDGCQRVKYGQCWVFAAVACTVLRCLGIPTRVVTNYNSAHDQNSNLLIEYFRNEFGEIQSDKSEMIWNFHCWVESWMTRPDLQPGYEGWQALDPTPQEKSEGTYCCGPVPVRAIKEGDLSTKYDAPFVFAEVNADVVDWIRQDDGSLHKSINHSLVVGLKISTKCVGRDDREDITHSYKYPEGSPEEREAFTRANHLNKLVNKEETGVAMRIRVGEGMNRGCDFDVFAHITNSTPEEHTGRLLLCARTVSYNGILGPECGTKDLLSLSLEPYSEKSIPLRILYEKYCDCLTESNLIKVRGLLIEPAANSYLLAERDIYLENPEIKIRILGEPKQNRKLVAEISLQNPLTVALSGCTFTVEGAGLIEEQKTVDVPDPVEAGEEVKVRVDLLPLYVGRHKLVVNFESDRLKAVKGFRNVIVGPSGEEV

SEQ ID NO: 24 - protein, 686 aa, Mouse
MAEELLLERCDLEIQANGRDHHTADLCQEKLVLRRGQRFRLTLYFEGRGYEASVDSLTFGAVTGPDPSEEAGTKARFSLSDNVEEGSWSASVLDQQDNVLSLQLCTPANAPIGLYRLSLEASTGYQGSSFVLGHFILLYNAWCPADDVYLDSEEERREYVLTQQGFIYQGSVKFIKSVPWNFGQFEDGILDTCLMLLDMNPKFLKNRSRDCSRRSSPIYVGRVVSAMVNCNDDQGVLLGRWDNNYGDGISPMAWIGSVDILRRWKEHGCQQVKYGQCWVFAAVACTVLRCLGIPTRVVTNYNSAHDQNSNLLIEYFRNEFGELESNKSEMIWNFHCWVESWMTRPDLQPGYEGWQAIDPTPQEKSEGTYCCGPVSVRAIKEGDLSTKYDAPFVFAEVNADVVDWIRQEDGSVLKSINRSLVVGQKISTKSVGRDDREDITHTYKYPEGSPEEREVFTKANHLNKLAEKEETGVAMRIRVGDSMSMGNDFDVFAHIGNDTSETRECRLLLCARTVSYNGVLGPECGTEDINLTLDPYSENSIPLRILYEKYSGCLTESNLIKVRGLLIEPAANSYLLAERDLYLENPEIKIRVLGEPKQNRKLVAEVSLKNPLSDPLYDCIFTVEGAGLTKEQKSVEVSDPVPAGDLVKARVDLFPTDIGLHKLVVNFQCDKLKSVKGYRNVIIGPA

SEQ ID NO: 25 - protein, 686 aa, Rat
MAEELNLERCDLEIQANGRDHHTADLCQQKLVLRRGQRFRLTLYFEGRGYEASVDRLTFGAVTGPDPSEEAGTKARFSLSDDVEEGSWSASVLDQQDNVLSLQLCTPANAPVGQYRLSLETSTGYQGSSFMLGHFILLFNAWCPADDVYLDSEAERREYVLTQQGFIYQGSVKFIKSVPWNFGQFEDGILDACLMLLDVNPKFLKDRSRDCSRRSSPIYVGRVVSGMVNCNDDQGVLLGRWDNNYGDGISPMAWIGSVDILRRWKEHGCQQVKYGQCWVFAAVACTVLRCLGIPTRVVTNYNSAHDQNSNLLIEYFRNEYGELESNKSEMIWNFHCWVESWMTRPDLQPGYEGWQAIDPTPQEKSEGTYCCGPVSVRAIKEGDLSTKYDASFVFAEVNADVVDWIRQSDGSVLKSINNSLVVGQKISTKSVGRDDREDITYTYKYPEGSPEEREVFTRANHLNKLAEKEETGVAMRIRVGDGMSLGNDFDVFAHIGNDTSESRECRLLLCARTVSYNGVLGPECGTEDINLTLDPYSENSIPLRILYEKYSGCLTESNLIKVRGLLVEPAANSYLLAERDLYLENPEIKIRILGEPKQNRKLVAEVSLKNPLSDSLYDCVFTVEGAGLTKEQKSVEVSDPVPAGDAVKVRVDLFPTDIGLHKLVVNFQCDKLKSVKGYRNIIIGPA

(19) United States
(12) Patent Application Publication
(10) Pub. No.: US 2005/0221302 A1
(43) Pub. Date: Oct. 6, 2005

(54) Tissue transglutaminase

(76) Inventors: Michael Powell, Cardiff (GB); Jadwiga Furmaniak, Cardiff (GB); Bernard Rees Smith, Cardiff (GB)

Correspondence Address: MERCHANT & GOULD PC, P.O. BOX 2903, MINNEAPOLIS, MN, 55402-0903, US

(21) Appl. No.: 10/467,587

(22) PCT Filed: Feb. 7, 2002
(86) PCT No.: PCT/GB02/00520

§ 371 Date: Jan. 29, 2004

(30) Foreign Application Priority Data
Feb. 7, 2001 (GB) 0103024.6

(51) Int. Cl. C12Q 1/68; C07H 21/04; C07K 16/42; C12N 5/06

(52) U.S. Cl. 435/6; 435/69.1; 435/327; 435/320.1; 530/387.2; 536/23.53

(57) ABSTRACT

The present invention relates to tissue transglutaminase (tTG), and more particularly to DNA coding for and polypeptides containing, the primary structural conformaion of one or more epitopes of tTG and to the use thereof in the detection of autoantibodies and/or lymphocytes produced in response to tTG.

The present invention relates to tissue transglutaminase (tTG), and more particularly to DNA coding for and polypeptides containing, the primary structural conformation of one or more epitopes of tTG and to the use thereof in the detection of autoantibodies and/or lymphocytes produced in response to tTG.

tTG belongs to the class of transglutaminases (TGs). The TGs are enzymes catalysing an acyl transfer depending on Ca²⁺, the γ-carboxamide groups of peptide-bonded glutamine residues acting as acyl donors. Primarily, protein-bonded lysine residues function as acyl acceptors, so that the transfer results in an ε-(γ-glutamyl)lysine bond. The substrate specificity of the TGs with respect to the acyl donors is very high (depending on the amino acid sequence), whereas an exceptionally wide spectrum of acceptors is available. The covalent peptide bonds formed are highly stable and protease-resistant, resulting in an increased resistance of the crosslinked proteins to chemical, enzymatic or physical effects.

There is a widespread occurrence of various TGs in miscellaneous organs, tissues, in plasma and interstitial body fluids, correlating with the occurrence of transglutaminase-modified proteins in blood clots, on cell membranes, in the horny layer of the epidermis, in hair, in nails, and in the extracellular matrix. The described TGs may be distinguished by their physical properties, their location in the body, and their primary structure.

tTG is also referred to as cellular, erythrocyte, endothelial, cytoplasmatic, liver, or type II TG, and is a monomer having a molecular weight of 75-85 kDa. The complete amino acid sequence comprising 687 residues has been derived from cDNA. At the protein level, there is an 84% homology between the human enzyme and the enzyme from mouse macrophages and an 81% homology between the human and guinea pig enzymes.

There are a number of diseases that may be associated with tTG and can include autoimmune diseases and inflammatory diseases. In particular, autoimmune diseases associated with an immune reaction to tTG can be autoimmune diseases arising due to gluten sensitive enteropathy, such as coeliac disease or sprue.

Coeliac disease is a disease of the small intestine mucosa, the first manifestation predominantly occurring during the late infant and toddler ages. If the corresponding clinical picture does not occur before the adult age, it is generally termed non-tropical sprue. Thus, both of these terms describe the same disease, which is hereinafter referred to as coeliac disease.

Coeliac disease is estimated to effect about 1 in 300 individuals in Western countries. The characteristic intolerance of dietary gluten present in wheat, barley, rye and related products results in an inflammatory disease of the upper small intestine in genetically susceptible individuals. Typical symptoms include chronic diarrhoea, abdominal distension and failure to thrive. In some patients symptoms of malabsorption such as anaemia and osteoporosis may manifest. Other symptoms include inflammatory lesions in the small bowel, for example, villous atrophy, hypertrophic crypts and increased number of inter-epithelial lymphocytes.

Provided the disease is diagnosed in time the symptoms can generally be treated by adhering to a gluten-free diet. However malabsorption may give rise to severe disease symptoms if the disease is not diagnosed and treated. Ultimately, there is the increased risk of developing an intestinal lymphoma and also other gastrointestinal neoplasias.

Diagnosis of coeliac disease is currently based on clinical symptoms, histological identification of gluten sensitive enteropathy, and serological tests for observing any response to withdrawal of gluten from an individual's diet. An example of a serological test used for diagnosis and monitoring of coeliac disease is an immunofluorescence test measuring endomysial antibody and measurement of autoantibodies to tTG.

WO98/03872 discloses that tTG is an autoantigen assocaited with coeliac disease and specifically discloses an ELISA based on guinea pig tTG of about 60% purity.

It would be advantageous to be able to non-invasively efficiently and reliably diagnose and monitor autoimmune diseases associated with tTG, such as coeliac disease as referred to above. For example, the above described immunofluorescence test measuring endomysial antibody requires special expertise and equipment and is dependent on subjective assessment of fluorescence patterns. It would, therefore, be advantageous to be able to replace the immunofluorescence test measuring endomysial antibody with a non-invasive sensitive and specific test for measuring autoantibodies to tTG.

It is, therefore, an aim of the present invention to provide an assay system, which alleviates some of the aforementioned problems arising in the diagnosis and monitoring of autoimmune diseases associated with an immune reaction to tTG. It is a further aim of the present invention to provide an assay for measuring autoantibodies or lymphocytes produced in response to tTG, with improved sensitivity and specificity and also provide diagnostic kits for use in the simple and rapid detection of autoantibodies or lymphocytes produced in response to tTG substantially as referred to above. A better understanding of tTG, and epitopes thereof, would be beneficial in achieving the above described aims of the present invention.

More particularly, we have now specifically identified that antigenic determinants (or epitopes) of tTG which interact with autoantibodies and/or lymphocytes produced in response to tTG, and present in sera from patients with coeliac disease, are located in the N-terminal and/or central part of the tTG molecule. In particular, our studies have shown that the N-terminal region (typically amino acids 1 to 89) and/or the central region (typically amino acids 401 to 494 or amino acids 401 to 491) of the tTG molecule are particularly important for interaction with autoantibodies and/or lymphocytes produced in response to tTG and present in sera from coeliac patients.

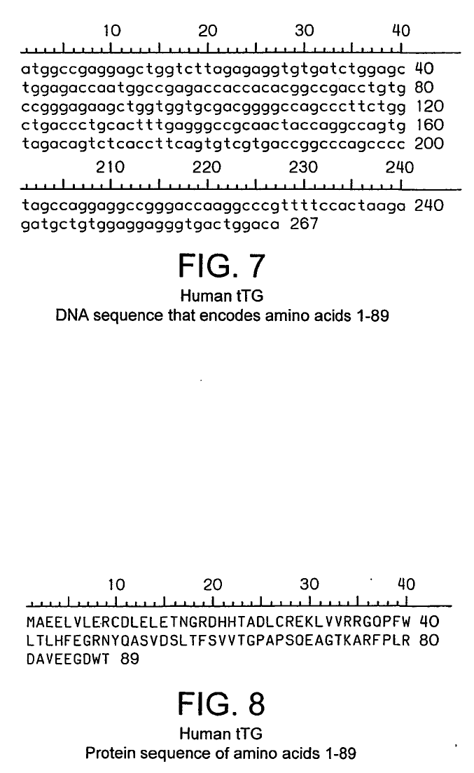

There is provided by the present invention, therefore, a DNA sequence encoding:
a polypeptide with which autoantibodies and/or lymphocytes produced in response to tTG can interact (under conditions that allow interaction of tTG with such autoantibodies or lymphocytes), and which comprises part or all of the primary structural conformation (that is a continuous sequence of amino acid residues) of one or more epitopes of tTG with which autoantibodies and/or lymphocytes produced in response to tTG can interact (under conditions that allow interaction of tTG with such autoantibodies or lymphocytes);
which DNA sequence comprises:
   (a) a DNA sequence as depicted in any of FIGS. 7, 9, 11, 13, 15, 17, 19, 21, 23 or 25;
   (b) a DNA sequence encoding a polypeptide comprising the primary structural conformation of amino acid numbers 1 to 89 of tTG, and/or the primary structural conformation of amino acid numbers 401 to 494 of tTG or the primary structural conformation of amino acid numbers 401 to 491 of tTG, or one or more active fragments of:
      amino acid numbers 1 to 89 of tTG, and/or amino acid numbers 401 to 494 of tTG or amino acid numbers 401 to 491 of tTG, with which autoantibodies and/or lymphocytes produced in response to tTG can interact (under conditions that allow interaction of tTG with such autoantibodies or lymphocytes),
   with the exception of human, guinea pig, mouse, bovine or rat full length tTG (or more generally full length tTG);
   (c) a DNA sequence encoding a polypeptide comprising the primary structural conformation of amino acids as depicted in one or more of FIGS. 8, 10, 12, 14, 16, 18, 20, 22, 24 and 26, or one or more active fragments of amino acids as depicted in one or more of FIGS. 8, 10, 12, 14, 16, 18, 20, 22, 24 and 26, with which autoantibodies and/or lymphocytes produced in response to tTG can interact (under conditions that allow interaction of tTG with such autoantibodies or lymphocytes), with the exception of human, guinea pig, mouse, bovine or rat full length tTG (or more generally full length tTG);
   (d) a DNA sequence differing from the DNA sequence of (a) in codon sequence due to the degeneracy of the genetic code;
   (e) a DNA sequence comprising a fragment or an allelic variation of the DNA sequence of (a); and
   (f) a DNA sequence which hybridizes to any of DNA sequences (a), (b), (c), (d) or (e) and encodes a polypeptide with which autoantibodies and/or lymphocytes produced in response to tTG can interact (under conditions that allow interaction of tTG with such autoantibodies or lymphocytes) and which comprises part or all of the primary structural conformation of one or more epitopes of tTG with which autoantibodies and/or lymphocytes produced in response to tTG can interact (under conditions that allow interaction of tTG with such autoantibodies or lymphocytes).

There is also provided by the present invention a DNA sequence encoding:
a polypeptide with which autoantibodies and/or lymphocytes produced in response to tTG can interact (under conditions that allow interaction of tTG with such autoantibodies or lymphocytes), and which comprises part or all of the primary structural conformation (that is a continuous sequence of amino acid residues) of one or more epitopes of tTG with which autoantibodies and/or lymphocytes produced in response to tTG can interact (under conditions that allow interaction of tTG with such autoantibodies or lymphocytes);
which DNA sequence comprises:
   (a) a DNA sequence as depicted in any of FIGS. 7, 9, 11, 13 or 15;
   (b) a DNA sequence encoding a polypeptide comprising the primary structural conformation of amino acid numbers 1 to 89 of tTG, or one or more active fragments of amino acid numbers 1 to 89 of tTG, with which autoantibodies and/or lymphocytes produced in response to tTG can interact (under conditions that allow interaction of tTG with such autoantibodies or lymphocytes), with the exception of human, guinea pig, mouse, bovine or rat full length tTG (or more generally full length tTG);
   (c) a DNA sequence encoding a polypeptide comprising the primary structural conformation of amino acids as depicted in one or more of FIGS. 8, 10, 12, 14 and 16, or one or more active fragments of amino acids as depicted in one or more of FIGS. 8, 10, 12, 14 and 16, with which autoantibodies and/or lymphocytes produced in response to tTG can interact (under conditions that allow interaction of tTG with such autoantibodies or lymphocytes), with the exception of human, guinea pig, mouse, bovine or rat full length tTG (or more generally full length tTG);
   (d) a DNA sequence differing from the DNA sequence of (a) in codon sequence due to the degeneracy of the genetic code;
   (e) a DNA sequence comprising a fragment or an allelic variation of the DNA sequence of (a); and
   (f) a DNA sequence which hybridizes to any of DNA sequences (a), (b), (c), (d) or (e) and encodes a polypeptide with which autoantibodies and/or lymphocytes produced in response to tTG can interact (under conditions that allow interaction of tTG with such autoantibodies or lymphocytes) and which comprises part or all of the primary structural conformation of one or more epitopes of tTG with which autoantibodies and/or lymphocytes produced in response to tTG can interact (under conditions that allow interaction of tTG with such autoantibodies or lymphocytes).

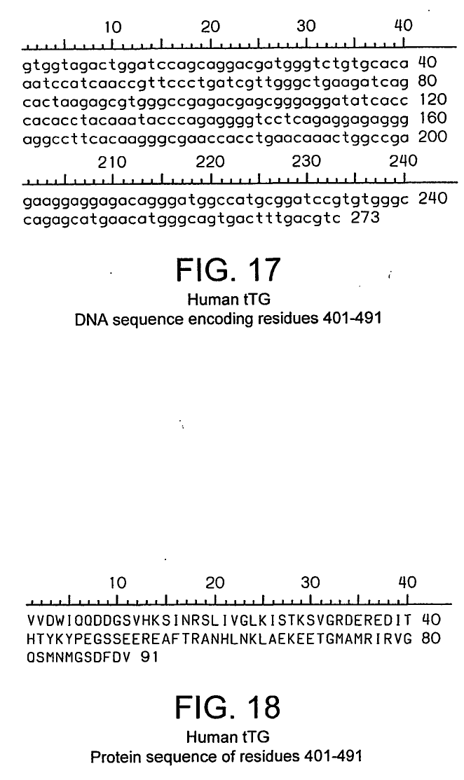

There is also provided by the present invention a DNA sequence encoding:
a polypeptide with which autoantibodies and/or lymphocytes produced in response to tTG can interact (under conditions that allow interaction of tTG with such autoantibodies or lymphocytes), and which comprises part or all of the primary structural conformation (that is a continuous sequence of amino acid residues) of one or more epitopes of tTG with which autoantibodies and/or lymphocytes produced in response to tTG can interact (under conditions that allow interaction of tTG with such autoantibodies or lymphocytes);
which DNA sequence comprises:
   (a) a DNA sequence as depicted in any of FIGS. 17, 19, 21, 23 or 25;
   (b) a DNA sequence encoding a polypeptide comprising the primary structural conformation of amino acid numbers 401 to 494 of tTG or the primary structural conformation of amino acid numbers 401 to 491 of tTG, or one or more active fragments of:
      amino acid numbers 401 to 494 of tTG or amino acid numbers 401 to 491 of tTG, with which autoantibodies and/or lymphocytes produced in response to tTG can interact (under conditions that allow interaction of tTG with such autoantibodies or lymphocytes),
   with the exception of human, guinea pig, mouse, bovine or rat full length tTG (or more generally full length tTG);
   (c) a DNA sequence encoding a polypeptide comprising the primary structural conformation of amino acids as depicted in one or more of FIGS. 18, 20, 22, 24 and 26, or one or more active fragments of amino acids as depicted in one or more of FIGS. 18, 20, 22, 24 and 26, with which autoantibodies and/or lymphocytes produced in response to tTG can interact (under conditions that allow interaction of tTG with such autoantibodies or lymphocytes), with the exception of human, guinea pig, mouse, bovine or rat full length tTG (or more generally full length tTG);
   (d) a DNA sequence differing from the DNA sequence of (a) in codon sequence due to the degeneracy of the genetic code;
   (e) a DNA sequence comprising a fragment or an allelic variation of the DNA sequence of (a); and
   (f) a DNA sequence which hybridizes to any of DNA sequences (a), (b), (c), (d) or (e) and encodes a polypeptide with which autoantibodies and/or lymphocytes produced in response to tTG can interact (under conditions that allow interaction of tTG with such autoantibodies or lymphocytes) and which comprises part or all of the primary structural conformation of one or more epitopes of tTG with which autoantibodies and/or lymphocytes produced in response to tTG can interact (under conditions that allow interaction of tTG with such autoantibodies or lymphocytes).

There is also provided by the present invention a DNA sequence encoding:
a polypeptide with which autoantibodies produced in response to tTG can interact (under conditions that allow interaction of tTG with such autoantibodies), and which comprises part or all of the primary structural conformation (that is a continuous sequence of amino acid residues) of one or more epitopes of tTG with which autoantibodies produced in response to tTG can interact (under conditions that allow interaction of tTG with such autoantibodies);
which DNA sequence comprises:
   (a) a DNA sequence as depicted in any of FIGS. 7, 9, 11, 13, 15, 17, 19, 21, 23 or 25;
   (b) a DNA sequence encoding a polypeptide comprising the primary structural conformation of amino acid numbers 1 to 89 of tTG, and/or the primary structural conformation of amino acid numbers 401 to 494 of tTG or the primary structural conformation of amino acid numbers 401 to 491 of tTG, or one or more active fragments of:
      amino acid numbers 1 to 89 of tTG, and/or amino acid numbers 401 to 494 of tTG or amino acid numbers 401 to 491 of tTG, with which autoantibodies produced in response to tTG can interact (under conditions that allow interaction of tTG with such autoantibodies),
   with the exception of human, guinea pig, mouse, bovine or rat full length tTG (or more generally full length tTG);
   (c) a DNA sequence encoding a polypeptide comprising the primary structural conformation of amino acids as depicted in one or more of FIGS. 8, 10, 12, 14, 16, 18, 20, 22, 24 and 26, or one or more active fragments of amino acids as depicted in one or more of FIGS. 8, 10, 12, 14, 16, 18, 20, 22, 24 and 26, with which autoantibodies produced in response to tTG can interact (under conditions that allow interaction of tTG with such autoantibodies), with the exception of human, guinea pig, mouse, bovine or rat full length tTG (or more generally full length tTG);
   (d) a DNA sequence differing from the DNA sequence of (a) in codon sequence due to the degeneracy of the genetic code;
   (e) a DNA sequence comprising a fragment or an allelic variation of the DNA sequence of (a); and
   (f) a DNA sequence which hybridizes to any of DNA sequences (a), (b), (c), (d) or (e) and encodes a polypeptide with which autoantibodies produced in response to tTG can interact (under conditions that allow interaction of tTG with such autoantibodies) and which comprises part or all of the primary structural conformation of one or more epitopes of tTG with which autoantibodies produced in response to tTG can interact (under conditions that allow interaction of tTG with such autoantibodies).

There is also provided by the present invention a DNA sequence encoding:
a polypeptide with which autoantibodies produced in response to tTG can interact (under conditions that allow interaction of tTG with such autoantibodies), and which comprises part or all of the primary structural conformation (that is a continuous sequence of amino acid residues) of one or more epitopes of tTG with which autoantibodies produced in response to tTG can interact (under conditions that allow interaction of tTG with such autoantibodies);
which DNA sequence comprises:
   (a) a DNA sequence as depicted in any of FIGS. 7, 9, 11, 13 or 15;
   (b) a DNA sequence encoding a polypeptide comprising the primary structural conformation of amino acid numbers 1 to 89 of tTG, or one or more active fragments of amino acid numbers 1 to 89 of tTG, with which autoantibodies produced in response to tTG can interact (under conditions that allow interaction of tTG with such autoantibodies),
   with the exception of human, guinea pig, mouse, bovine or rat full length tTG (or more generally full length tTG);
   (c) a DNA sequence encoding a polypeptide comprising the primary structural conformation of amino acids as depicted in one or more of FIGS. 8, 10, 12, 14 and 16, or one or more active fragments of amino acids as depicted in one or more of FIGS. 8, 10, 12, 14 and 16, with which autoantibodies produced in response to tTG can interact (under conditions that allow interaction of tTG with such autoantibodies), with the exception of human, guinea pig, mouse, bovine or rat full length tTG (or more generally full length tTG);
   (d) a DNA sequence differing from the DNA sequence of (a) in codon sequence due to the degeneracy of the genetic code;
   (e) a DNA sequence comprising a fragment or an allelic variation of the DNA sequence of (a); and
   (f) a DNA sequence which hybridizes to any of DNA sequences (a), (b), (c), (d) or (e) and encodes a polypeptide with which autoantibodies produced in response to tTG can interact (under conditions that allow interaction of tTG with such autoantibodies) and which comprises part or all of the primary structural conformation of one or more epitopes of tTG with which autoantibodies produced in response to tTG can interact (under conditions that allow interaction of tTG with such autoantibodies).

There is also provided by the present invention a DNA sequence encoding:
a polypeptide with which autoantibodies produced in response to tTG can interact (under conditions that allow interaction of tTG with such autoantibodies), and which comprises part or all of the primary structural conformation (that is a continuous sequence of amino acid residues) of one or more epitopes of tTG with which autoantibodies produced in response to tTG can interact (under conditions that allow interaction of tTG with such autoantibodies);
which DNA sequence comprises:
   (a) a DNA sequence as depicted in any of FIGS. 17, 19, 21, 23 or 25;
   (b) a DNA sequence encoding a polypeptide comprising the primary structural conformation of amino acid numbers 401 to 494 of tTG or the primary structural conformation of amino acid numbers 401 to 491 of tTG, or one or more active fragments of:
      amino acid numbers 401 to 494 of tTG or amino acid numbers 401 to 491 of tTG, with which autoantibodies produced in response to tTG can interact (under conditions that allow interaction of tTG with such autoantibodies),
   with the exception of human, guinea pig, mouse, bovine or rat full length tTG (or more generally full length tTG);
   (c) a DNA sequence encoding a polypeptide comprising the primary structural conformation of amino acids as depicted in one or more of FIGS. 18, 20, 22, 24 and 26, or one or more active fragments of amino acids as depicted in one or more of FIGS. 18, 20, 22, 24 and 26, with which autoantibodies produced in response to tTG can interact (under conditions that allow interaction of tTG with such autoantibodies), with the exception of human, guinea pig, mouse, bovine or rat full length tTG (or more generally full length tTG);
   (d) a DNA sequence differing from the DNA sequence of (a) in codon sequence due to the degeneracy of the genetic code;
   (e) a DNA sequence comprising a fragment or an allelic variation of the DNA sequence of (a); and
   (f) a DNA sequence which hybridizes to any of DNA sequences (a), (b), (c), (d) or (e) and encodes a polypeptide with which autoantibodies produced in response to tTG can interact (under conditions that allow interaction of tTG with such autoantibodies) and which comprises part or all of the primary structural conformation of one or more epitopes of tTG with which autoantibodies produced in response to tTG can interact (under conditions that allow interaction of tTG with such autoantibodies).

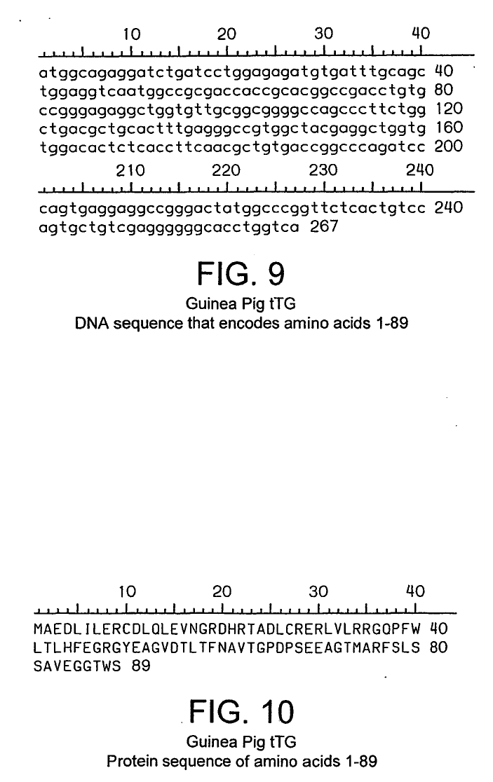

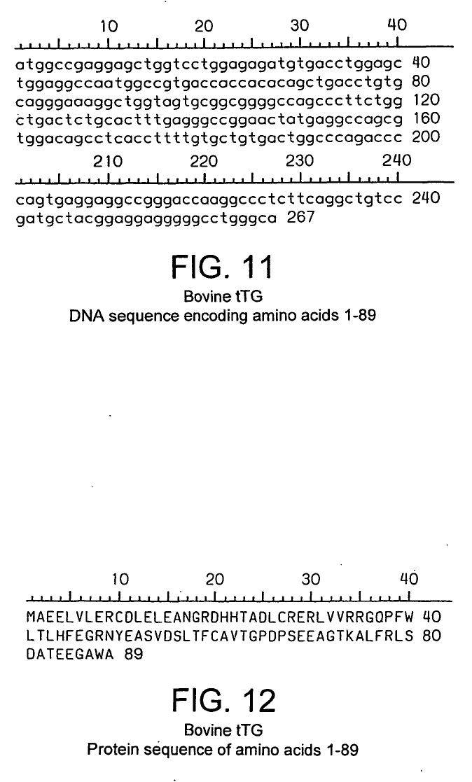

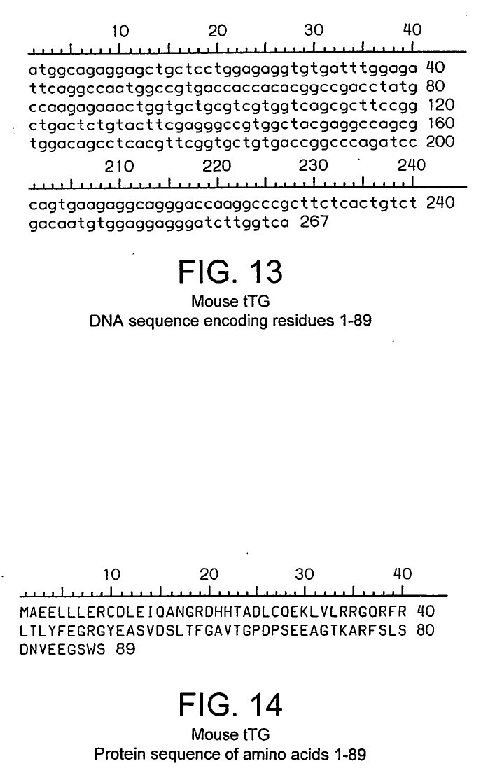

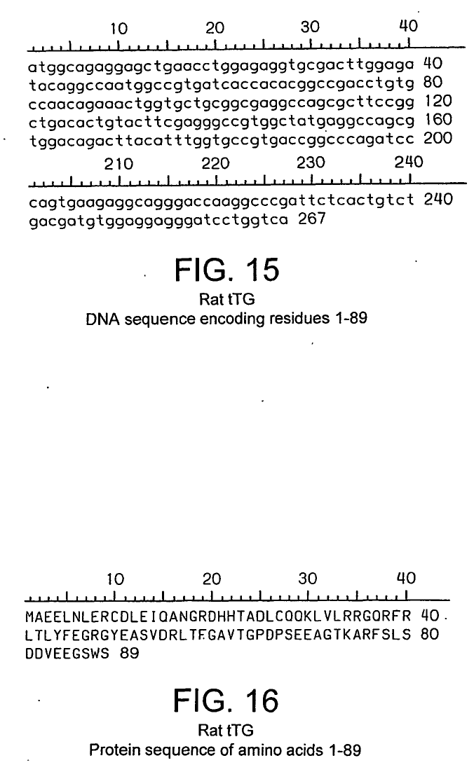

A DNA sequence as provided by the present invention can be of human origin (as depicted in FIGS. 7 and 77), of guinea pig origin (as depicted in FIGS. 9 and 99), of bovine origin (as depicted in FIGS. 11 and 11), of mouse origin (as depicted in FIGS. 13 and 33), or of rat origin (as depicted in FIGS. 15 and 55). In particular, the present invention provides a DNA sequence as depicted in any of FIGS. 7, 9, 11, 13, 15, 17, 19, 21, 23 or 25, or a DNA sequence differing therefrom due to the degeneracy of the genetic code.

More particularly, a DNA sequence as provided by the present invention can encode part or all of the primary structural conformation of amino acid numbers 1 to 89 of tTG, and/or part or all of the primary structural conformation of amino acid numbers 401 to 494 of tTG or part or all of the primary structural conformation of amino acid numbers 401 to 491 of tTG substantially as hereinafter described in greater detail.

A DNA sequence as provided by the present invention can be included in a biologically functional plasmid or DNA vector for expressing a polypeptide according to the present invention substantially as hereinafter described in greater detail and there is further provided by the present invention a biologically functional plasmid or DNA vector including a DNA sequence substantially as hereinbefore described. Furthermore, there is still further provided by the present invention a host cell which is transformed or transfected with a DNA sequence substantially as hereinbefore described, or a plasmid or vector substantially as hereinbefore described. A suitable host cell can be any prokaryote or eukaryote cell capable of replicating and expressing a DNA sequence substantially as hereinbefore described and suitable techniques are well known to one of ordinary skill in the art.

Polypeptides according to the present invention substantially as herein described can be expressed in various systems generating recombinant proteins. For example, for expression in E coli, cDNA coding for the appropriate polypeptides according to the present invention can be cloned into a vector, such as pMEX8, pGEXcT or pQE-81L His or an equivalent. In the case of expression in yeast (for example Saccharomyces cerevisiae or Schizosaccharomyces pombe), vectors such as pYES2, pESP2 or pYES2/CT or an equivalent, can be employed. AcMNPV (Bac-N-Blue) vector or an equivalent can be used for expression in insect cells and pRC/CMV vector or an equivalent can be used for expression in mammalian cells, such as Chinese Hamster Ovary (CHO) cells. A polypeptide according to the present invention can be expressed as a discrete protein, or as a fusion protein linked to, for example, glutathione S transferase (GST) or poly histidine linker. For a discrete protein, affinity column chromatography purification using a mouse monoclonal antibody to the relevant part of tTG coupled to a Sepharose particle can be used. If a tTG fragment is fused to GST, glutathione Sepharose chromatography purification can be used to isolate the fusion protein. Specific proteases can be used to separate GST from tTG peptide and a second round of glutathione Sepharose chromatography can be used to separate GST from a tTG fragment. In the case of peptides linked to poly histidine linker, the purification can be carried out using immobilised metal affinity chromatography.

There is also provided by the present invention a polypeptide with which autoantibodies and/or lymphocytes produced in response to tTG can interact (under conditions that allow interaction of tTG with such autoantibodies or lymphocytes) and which comprises part or all of the primary structural conformation (that is a continuous sequence of amino acid residues) of one or more epitopes of tTG with which autoantibodies and/or lymphocytes produced in response to tTG can interact (under conditions that allow interaction of tTG with such autoantibodies or lymphocytes), which polypeptide comprises the primary structural conformation of amino acid numbers 1 to 89 of tTG, and/or the primary structural conformation of amino acid numbers 401 to 494 of tTG or the primary structural conformation of amino acid numbers 401 to 491 of tTG, or one or more active fragments of:
amino acid numbers 1 to 89 of tTG, and/or amino acid numbers 401 to 494 of tTG or amino acid numbers 401 to 491 of tTG, with which autoantibodies and/or lymphocytes produced in response to tTG can interact (under conditions that allow interaction of tTG with such autoantibodies or lymphocytes);

with the exception of human, guinea pig, mouse, bovine or rat full length tTG (or more generally full length Ttg).

There is also provided by the present invention a polypeptide with which autoantibodies and/or lymphocytes produced in response to tTG can interact (under conditions that allow interaction of tTG with such autoantibodies or lymphocytes) and which comprises part or all of the primary structural conformation (that is a continuous sequence of amino acid residues) of one or more epitopes of tTG with which autoantibodies and/or lymphocytes produced in response to tTG can interact (under conditions that allow interaction of tTG with such autoantibodies or lymphocytes), which polypeptide comprises the primary structural conformation of amino acid numbers 1 to 89 of tTG, or one or more active fragments of amino acid numbers 1 to 89 of tTG, with which autoantibodies and/or lymphocytes produced in response to tTG can interact (under conditions that allow interaction of tTG with such autoantibodies or lymphocytes), with the exception of human, guinea pig, mouse, bovine or rat full length tTG (or more generally full length tTG).

There is also provided by the present invention a polypeptide with which autoantibodies and/or lymphocytes produced in response to tTG can interact (under conditions that allow interaction of tTG with such autoantibodies or lymphocytes) and which comprises part or all of the primary structural conformation (that is a continuous sequence of amino acid residues) of one or more epitopes of tTG with which autoantibodies and/or lymphocytes produced in response to tTG can interact (under conditions that allow interaction of tTG with such autoantibodies or lymphocytes), which polypeptide comprises the primary structural conformation of amino acid numbers 401 to 494 of tTG or the primary structural conformation of amino acid numbers 401 to 491 of tTG, or one or more active fragments of:
amino acid numbers 401 to 494 of tTG or amino acid numbers 401 to 491 of tTG, with which autoantibodies and/or lymphocytes produced in response to tTG can interact (under conditions that allow interaction of tTG with such autoantibodies or lymphocytes);

with the exception of human, guinea pig, mouse, bovine or rat full length tTG (or more generally full length tTG).

There is also provided by the present invention a polypeptide with which autoantibodies produced in response to tTG can interact (under conditions that allow interaction of tTG with such autoantibodies) and which comprises part or all of the primary structural conformation (that is a continuous sequence of amino acid residues) of one or more epitopes of tTG with which autoantibodies produced in response to tTG can interact (under conditions that allow interaction of tTG with such autoantibodies), which polypeptide comprises the primary structural conformation of amino acid numbers 1 to 89 of tTG, and/or the primary structural conformation of amino acid numbers 401 to 494 of tTG or the primary structural conformation of amino acid numbers 401 to 491 of tTG, or one or more active fragments of:
amino acid numbers 1 to 89 of tTG, and/or amino acid numbers 401 to 494 of tTG or amino acid numbers 401 to 491 of tTG, with which autoantibodies produced in response to tTG can interact (under conditions that allow interaction of tTG with such autoantibodies);

with the exception of human, guinea pig, mouse, bovine or rat full length tTG (or more generally full length tTG).

There is also provided by the present invention a polypeptide with which autoantibodies produced in response to tTG can interact (under conditions that allow interaction of tTG with such autoantibodies) and which comprises part or all of the primary structural conformation (that is a continuous sequence of amino acid residues) of one or more epitopes of tTG with which autoantibodies produced in response to tTG can interact (under conditions that allow interaction of tTG with such autoantibodies), which polypeptide comprises the primary structural conformation of amino acid numbers 1 to 89 of tTG, or one or more active fragments of amino acid numbers 1 to 89 of tTG, with which autoantibodies produced in response to tTG can interact (under conditions that allow interaction of tTG with such autoantibodies), with the exception of human, guinea pig, mouse, bovine or rat full length tTG (or more generally full length tTG).

There is also provided by the present invention a polypeptide with which autoantibodies produced in response to tTG can interact (under conditions that allow interaction of tTG with such autoantibodies) and which comprises part or all of the primary structural conformation (that is a continuous sequence of amino acid residues) of one or more epitopes of tTG with which autoantibodies produced in response to tTG can interact (under conditions that allow interaction of tTG with such autoantibodies), which polypeptide comprises the primary structural conformation of amino acid numbers 401 to 494 of tTG or the primary structural conformation of amino acid numbers 401 to 491 of tTG, or one or more active fragments of:
amino acid numbers 401 to 494 of tTG or amino acid numbers 401 to 491 of tTG, with which autoantibodies produced in response to tTG can interact (under conditions that allow interaction of tTG with such autoantibodies);

with the exception of human, guinea pig, mouse, bovine or rat full length tTG (or more generally full length tTG).

The present invention further provides a polypeptide with which autoantibodies and/or lymphocytes produced in response to tTG can interact (under conditions that allow interaction of tTG with such autoantibodies or lymphocytes) and which comprises part or all of the primary structural conformation of one or more epitopes of tTG with which autoantibodies and/or lymphocytes produced in response to tTG can interact (under conditions that allow interaction of tTG with such autoantibodies or lymphocytes), which polypeptide comprises the primary structural conformation of amino acids as depicted in one or more of FIGS. 8, 10, 12, 14, 16, 18, 20, 22, 24 and 26, or one or more active fragments of amino acids as depicted in one or more of FIGS. 8, 10, 12, 14, 16, 18, 20, 22, 24 and 26, with which autoantibodies and/or lymphocytes produced in response to tTG can interact (under conditions that allow interaction of tTG with such autoantibodies or lymphocytes), with the exception of human, guinea pig, mouse, bovine or rat full length tTG (or more generally full length tTG).

The present invention further provides a polypeptide with which autoantibodies and/or lymphocytes produced in response to tTG can interact (under conditions that allow interaction of tTG with such autoantibodies or lymphocytes) and which comprises part or all of the primary structural conformation of one or more epitopes of tTG with which autoantibodies and/or lymphocytes produced in response to tTG can interact (under conditions that allow interaction of tTG with such autoantibodies or lymphocytes), which polypeptide comprises the primary structural conformation of amino acids as depicted in one or more of FIGS. 8, 10, 12, 14 and 16, or one or more active fragments of amino acids as depicted in one or more of FIGS. 8, 10, 12, 14 and 16, with which autoantibodies and/or lymphocytes produced in response to tTG can interact (under conditions that allow interaction of tTG with such autoantibodies or lymphocytes), with the exception of human, guinea pig, mouse, bovine or rat full length tTG (or more generally full length tTG).

The present invention further provides a polypeptide with which autoantibodies and/or lymphocytes produced in response to tTG can interact (under conditions that allow interaction of tTG with such autoantibodies or lymphocytes) and which comprises part or all of the primary structural conformation of one or more epitopes of tTG with which autoantibodies and/or lymphocytes produced in response to tTG can interact (under conditions that allow interaction of tTG with such autoantibodies or lymphocytes), which polypeptide comprises the primary structural conformation of amino acids as depicted in one or more of FIGS. 18, 20, 22, 24 and 26, or one or more active fragments of amino acids as depicted in one or more of FIGS. 18, 20, 22, 24 and 26, with which autoantibodies and/or lymphocytes produced in response to tTG can interact (under conditions that allow interaction of tTG with such autoantibodies or lymphocytes), with the exception of human, guinea pig, mouse, bovine or rat full length tTG (or more generally full length tTG).

The present invention further provides a polypeptide with which autoantibodies produced in response to tTG can interact (under conditions that allow interaction of tTG with such autoantibodies) and which comprises part or all of the primary structural conformation of one or more epitopes of tTG with which autoantibodies produced in response to tTG can interact (under conditions that allow interaction of tTG with such autoantibodies), which polypeptide comprises the primary structural conformation of amino acids as depicted in one or more of FIGS. 8, 10, 12, 14, 16, 18, 20, 22, 24 and 26, or one or more active fragments of amino acids as depicted in one or more of FIGS. 8, 10, 12, 14, 16, 18, 20, 22, 24 and 26, with which autoantibodies produced in response to tTG can interact (under conditions that allow interaction of tTG with such autoantibodies), with the exception of human, guinea pig, mouse, bovine or rat full length tTG (or more generally full length tTG).

The present invention further provides a polypeptide with which autoantibodies produced in response to tTG can interact (under conditions that allow interaction of tTG with such autoantibodies) and which comprises part or all of the primary structural conformation of one or more epitopes of tTG with which autoantibodies produced in response to tTG can interact (under conditions that allow interaction of tTG with such autoantibodies), which polypeptide comprises the primary structural conformation of amino acids as depicted in one or more of FIGS. 8, 10, 12, 14 and 16, or one or more active fragments of amino acids as depicted in one or more of FIGS. 8, 10, 12, 14 and 16, with which autoantibodies produced in response to tTG can interact (under conditions that allow interaction of tTG with such autoantibodies), with the exception of human, guinea pig, mouse, bovine or rat full length tTG (or more generally full length tTG).

The present invention further provides a polypeptide with which autoantibodies produced in response to tTG can interact (under conditions that allow interaction of tTG with such autoantibodies) and which comprises part or all of the primary structural conformation of one or more epitopes of tTG with which autoantibodies produced in response to tTG can interact (under conditions that allow interaction of tTG with such autoantibodies), which polypeptide comprises the primary structural conformation of amino acids as depicted in one or more of FIGS. 18, 20, 22, 24 and 26, or one or more active fragments of amino acids as depicted in one or more of FIGS. 18, 20, 22, 24 and 26, with which autoantibodies produced in response to tTG can interact (under conditions that allow interaction of tTG with such autoantibodies), with the exception of human, guinea pig, mouse, bovine or rat full length tTG (or more generally full length tTG).

A polypeptide as provided by the present invention can be of human origin (as depicted in FIGS. 8 and 88), of guinea pig origin (as depicted in FIGS. 10 and 20), of bovine origin (as depicted in FIGS. 12 and 22), of mouse origin (as depicted in FIGS. 14 and 44), or of rat origin (as depicted in FIGS. 16 and 66). In particular, the present invention provides a polypeptide as depicted in any of FIGS. 8, 10, 12, 14, 16, 18, 20, 22, 24 or 26.

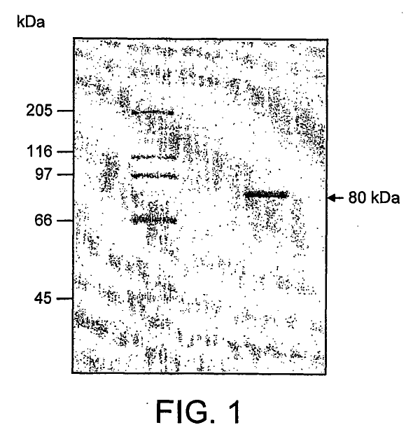

Typically, a polypeptide according to the present invention is a synthetic polypeptide derived by recombinant techniques substantially as herein described and is most suitably derived from recombinant human tTG typically with at least 70% purity, more preferably at least 90% purity and even more preferably at least 95% purity. Purity may be assessed by SDS PAGE analysis and Coomassie Blue staining as hereinafter discussed in connection with FIG. 1. The recombinant tTG (typically human) may be purified using affinity chromatography with tTG antibodies such as mouse tTG monoclonal antibody.

A polypeptide according to the present invention is preferably obtained by, or is obtainable by, expression of a DNA sequence according to the present invention substantially as hereinbefore described. A polypeptide according to the present invention obtained by such expression can be advantageous in being free from association with other eukaryotic polypeptides or contaminants which might otherwise be associated with tTG in its natural environment.

The present invention further provides a process of preparing a polypeptide substantially as hereinbefore described, which process comprises:
(i) providing a host cell substantially as hereinbefore described;
(ii) growing the host cell; and
(iii) recovering a polypeptide according to the present invention therefrom.

Recovery of a polypeptide according to the present invention can typically employ conventional isolation and purification techniques, such as chromatographic separations or immunological separations, known to one of ordinary skill in the art.

It is of course appreciated that there is provided within the scope of the present invention, one or more amino acid sequences or fragments thereof structurally distinct from the specific amino acid sequences of a polypeptide described herein (for example by the addition, deletion, substitution or insertion of one or more amino acids) but having substantially the same functional binding properties as the specific amino acid sequences of a polypeptide described herein. In other words, the present invention clearly encompasses within its scope amino acid sequences or fragments thereof substantially homologous to specific amino acid sequences of a polypeptide described herein, or representing variants of specific amino acid sequences of a polypeptide described herein, but having substantially the same functional binding properties as the specific amino acid sequences of a polypeptide described herein.

A polypeptide according to the present invention can in a first preferred embodiment of the present invention be a polypeptide with which autoantibodies and/or lymphocytes produced in response to tTG can interact (under conditions that allow interaction of tTG with such autoantibodies or lymphocytes) and which comprises part or all of the primary structural conformation (that is a continuous sequence of amino acid residues) of one or more epitopes of tTG with which autoantibodies and/or lymphocytes produced in response to tTG can interact (under conditions that allow interaction of tTG with such autoantibodies or lymphocytes), which polypeptide consists essentially of (or consists of) the primary structural conformation of amino acid numbers 1 to 89 of tTG, and/or the primary structural conformation of amino acid numbers 401 to 494 of tTG or the primary structural conformation of amino acid numbers 401 to 491 of tTG, or more particularly consists essentially of (or consists of) the primary structural conformation of amino acids as depicted in one or more of FIGS. 8, 10, 12, 14, 16, 18, 20, 22, 24 or 26, substantially as hereinbefore described.

Alternatively, in a second preferred embodiment of the present invention there is provided a polypeptide with which autoantibodies and/or lymphocytes produced in response to tTG can interact (under conditions that allow interaction of tTG with such autoantibodies or lymphocytes) and which comprises part or all of the primary structural conformation (that is a continuous sequence of amino acid residues) of one or more epitopes of tTG with which autoantibodies and/or lymphocytes produced in response to tTG can interact (under conditions that allow interaction of tTG with such autoantibodies or lymphocytes), which polypeptide consists essentially of (or consists of) one or more active fragments of amino acid numbers 1 to 89 of tTG, and/or one or more active fragments of amino acid numbers 401 to 494 of tTG or one or more active fragments of amino acid numbers 401 to 491 of tTG, with which autoantibodies and/or lymphocytes produced in response to tTG can interact (under conditions that allow interaction of tTG with such autoantibodies or lymphocytes), or more particularly consists essentially of (or consists of) the primary structural conformation of one or more active fragments of amino acids as depicted in one or more of FIGS. 8, 10, 12, 14, 16, 18, 20, 22, 24 or 26, with which autoantibodies and/or lymphocytes produced in response to tTG can interact (under conditions that allow interaction of tTG with such autoantibodies or lymphocytes) substantially as hereinbefore described. For example, a polypeptide consisting essentially of (or consisting of) amino acid numbers 1 to 85 of tTG, amino acid numbers 5 to 89 of tTG, or the like, with which autoantibodies and/or lymphocytes produced in response to tTG can interact (under conditions that allow interaction of tTG with such autoantibodies or lymphocytes).

A still further alternative in a third preferred embodiment of the present invention is a polypeptide (in particular a synthetic polypeptide, such as a scaffold type polypeptide typically providing amino acids corresponding to the amino acids of tTG as described herein in a form specifically adapted for use in an assay method or kit according to the present invention substantially as hereinafter described in greater detail) with which autoantibodies and/or lymphocytes produced in response to tTG can interact (under conditions that allow interaction of tTG with such autoantibodies or lymphocytes) and which includes part or all of the primary structural conformation of one or more epitopes of tTG with which autoantibodies and/or lymphocytes produced in response to tTG can interact (under conditions that allow interaction of tTG with such autoantibodies or lymphocytes), which polypeptide includes amino acids comprising the primary structural conformation of amino acid numbers 1 to 89 of tTG, and/or the primary structural conformation of amino acid numbers 401 to 494 of tTG or the primary structural conformation of amino acid numbers 401 to 491 of tTG, or one or more active fragments of amino acid numbers 1 to 89 of tTG, and/or one or more active fragments of amino acid numbers 401 to 494 of tTG or one or more active fragments of amino acid numbers 401 to 491 of tTG, with which autoantibodies and/or lymphocytes produced in response to tTG can interact (under conditions that allow interaction of tTG with such autoantibodies or lymphocytes); or more particularly which includes amino acids comprising the primary structural conformation of amino acids as depicted in one or more of FIGS. 8, 10, 12, 14, 16, 18, 20, 22, 24 or 26, or one or more active fragments of amino acids as depicted in one or more of FIGS. 8, 10, 12, 14, 16, 18, 20, 22, 24 or 26, with which autoantibodies and/or lymphocytes produced in response to tTG can interact (under conditions that allow interaction of tTG with such autoantibodies or lymphocytes), with the exception of human, guinea pig, mouse, bovine or rat full length tTG (or more generally full length tTG).

According to a particularly preferred embodiment of the present invention, there is also provided a method of screening for autoantibodies or lymphocytes produced in response to tTG in a sample of body fluid obtained from a subject (in particular a human) suspected of suffering from, susceptible to, having or recovering from autoimmune disease associated with an immune reaction to tTG, said method comprising:
(a) providing either (i) said sample of body fluid from said subject or (ii) lymphocytes isolated from said sample;
(b) contacting said sample or isolated lymphocytes with a polypeptide according to the present invention substantially as hereinbefore described (under conditions that allow interaction of tTG with autoantibodies or lymphocytes produced in response to tTG) so as to permit said polypeptide to interact with autoantibodies, or lymphocytes, produced in response to tTG, and present in, or isolated from, said sample; and
(c) monitoring the degree, or effect, of interaction of said polypeptide with either said autoantibodies, or said lymphocytes, produced in response to tTG and present in, or isolated from, said sample, thereby providing an indication of the presence of said autoantibodies, or said lymphocytes, in said sample, or isolated from said sample.

Substantially as described above, a method according to the present invention is suitable for screening for autoantibodies or lymphocytes produced in response to tTG in a sample of body fluid obtained from a subject. A method according to the present invention can, however, be particularly adapted for use in screening for autoantibodies produced in response to tTG in a sample of body fluid obtained from a subject substantially as hereinafter described in greater detail.

There is in particular provided by the present invention, therefore, a method of screening for autoantibodies produced in response to tTG in a sample of body fluid obtained from a subject (in particular a human) suspected of suffering from, susceptible to, having or recovering from autoimmune disease associated with an immune reaction to tTG, said method comprising:
(a) providing said sample of body fluid from said subject;
(b) contacting said sample with a polypeptide according to the present invention substantially as hereinbefore described (under conditions that allow interaction of tTG with autoantibodies produced in response to tTG) so as to permit said polypeptide to interact with autoantibodies produced in response to tTG and present in said sample; and
(c) monitoring the degree of interaction of said polypeptide with said autoantibodies produced in response to tTG and present in said sample, thereby providing an indication of the presence of said autoantibodies in said sample.

A method according to the present invention may typically employ a control, such as a sample of body fluid from a normal subject, in other words a subject known to be without autoimmune disease associated with an immune reaction to tTG.

A method of screening for autoantibodies to tTG according to the present invention may comprise directly monitoring interaction of (i) autoantibodies to tTG present in the sample of body fluid from the subject and (ii) a polypeptide, as provided by the present invention substantially as hereinbefore described, typically by employing non-competitive sandwich type assay techniques known in the art.

Typically, in a method according to the present invention employing non-competitive techniques, monitoring of the degree of interaction of (i) autoantibodies to tTG present in the sample and (ii) a polypeptide according to the present invention substantially as hereinbefore described, can comprise providing labelling means either to a polypeptide according to the present invention substantially as hereinbefore described, or to a binding partner for autoantibodies to tTG, either of which technique would enable monitoring of the above described interaction. For example, a method according to the present invention may comprise directly or indirectly labelling a polypeptide according to the present invention substantially as hereinbefore described; contacting the thus labelled polypeptide with a sample of body fluid being screened for tTG autoantibodies so as to provide a mixture thereof; and adding to the mixture a binding partner for autoantibodies to tTG (such as an anti-IgG reagent) present in the sample of body fluid, so as to cause precipitation of any complexes of labelled polypeptide and tTG autoantibodies present in the mixture. Alternatively, it may be preferred that a method according to the present invention further comprises adding a labelled binding partner for tTG autoantibodies (such as a labelled anti-IgG reagent, for example protein A or anti-human IgG, or labelled full length tTG or an epitope thereof) to a mixture obtained by contacting (i) a polypeptide according to the present invention substantially as hereinbefore described immobilised to a support and (ii) a sample of body fluid being screened for autoantibodies to tTG.

It may alternatively be preferred that a method of screening for autoantibodies to tTG in the sample of body fluid according to the present invention, utilises the principles employed in known competitive assays. For example, a method according to the present invention may employ at least one competitor capable of competing with autoantibodies to tTG in the interaction thereof with a polypeptide according to the present invention substantially as hereinbefore described.

Typically, a competitor as employed in a competitive assay method according to the present invention may comprise one or more antibodies, which may be natural or partly or wholly synthetically produced. A competitor as employed in the present invention may alternatively comprise any other protein having a binding domain or region which is capable of competing with autoantibodies to tTG in the interaction thereof with a polypeptide according to the present invention substantially as hereinbefore described. Preferably, however, a competitor as employed in the present invention comprises a monoclonal, recombinant or polyclonal antibody (especially a monoclonal antibody), capable of competing with tTG autoantibodies in the interaction thereof with a polypeptide according to the present invention substantially as hereinbefore described.

Typically, therefore, a competitive assay method according to the present invention may further comprise providing at least one competitor, such as a monoclonal or polyclonal antibody, whereby in step (b) of a method as herein described a polypeptide according to the present invention substantially as hereinbefore described can interact with either a competitor, such as a monoclonal or polyclonal antibody, or autoantibodies to tTG present in said sample.

Typically monitoring in a competitive assay method according to the present invention comprises comparing:
(i) interaction of a polypeptide according to the present invention substantially as hereinbefore described and one or more competitors substantially as hereinbefore described (typically a monoclonal or polyclonal antibody), in the absence of said sample of body fluid being screened (that is a suspected disease sample), optionally in the presence of a sample of body fluid from a normal subject, typically a subject known to be without autoimmune disease associated with an immune reaction to tTG; with
(ii) interaction of a polypeptide according to the present invention substantially as hereinbefore described and one or more competitors substantially as hereinbefore described (typically a monoclonal or polyclonal antibody), in the presence of said sample of body fluid being screened.

Typically, the comparison involves observing a decrease in interaction of a polypeptide according to the present invention substantially as hereinbefore described and the competitor in (ii) compared to (i) so as to provide an indication of the presence of autoantibodies to tTG in said sample. Typically, the decrease in interaction can be observed by directly or indirectly labelling the competitor and monitoring any change in the interaction of the thus labelled competitor with a polypeptide according to the present invention substantially as hereinbefore described in the absence and in the presence of a sample of body fluid being screened for autoantibodies to tTG. Suitably a polypeptide according to the present invention substantially as hereinbefore described may be immobilised to facilitate the above mentioned monitoring.

Alternatively, there is also provided by the present invention a method of screening for autoantibodies to tTG in a sample of body fluid obtained from a subject (in particular a human) suspected of suffering from, susceptible to, having or recovering from autoimmune disease associated with an immune reaction to tTG, said method comprising:
(a) providing said sample of body fluid from said subject;
(b) contacting said sample with
   (i) full length tTG (typically recombinantly obtained full length tTG), and
   (ii) at least one competitor capable of competing with autoantibodies to tTG in the interaction thereof with a polypeptide according to the present invention substantially as hereinbefore described,
   (under conditions that allow interaction of tTG with autoantibodies to tTG), so as to permit said full length tTG to interact with either autoantibodies to tTG present in said sample, or said competitor; and
(c) monitoring the interaction of said full length tTG with said autoantibodies present in said sample, thereby providing an indication of the presence of said autoantibodies to tTG in said sample.

The full length tTG can typically be of human, guinea pig, bovine, mouse or rat origin, preferably human, and more preferably human recombinantly obtained full length tTG. A competitor for use in such an assay typically comprises a monoclonal or polyclonal antibody (preferably monoclonal) substantially as hereinbefore described.

Suitably a detectable label that can be employed in a method according to the present invention can be selected from the group consisting of enzymic labels, isotopic labels, chemiluminescent labels, fluorescent labels, dyes and the like.

In the case where an isotopic label (such as ¹²⁵I, ¹⁴C, ³H or ³⁵S) is employed, monitoring may therefore comprise measuring radioactivity dependent on binding of a polypeptide according to the present invention substantially as hereinbefore described. Radioactivity is generally measured using a gamma counter, or liquid scintillation counter.

In the case of a method of screening for lymphocytes according to the present invention, it is generally preferred that lymphocytes are initially isolated from a sample of body fluid from a subject using techniques well known to one of ordinary skill in the art, followed by contact with a polypeptide according to the present invention so as to stimulate the proliferation of the isolated lymphocytes. Monitoring of the effect of interaction of a polypeptide according to the present invention and such proliferating lymphocytes, typically employs means known in the art for monitoring such proliferation of lymphocytes.

According to a further particularly preferred embodiment of the present invention, there is provided a kit for screening for autoantibodies or lymphocytes produced in response to tTG in a sample of body fluid obtained from a subject (in particular a human) suspected of suffering from, susceptible to, having or recovering from autoimmune disease associated with an immune reaction to tTG, said kit comprising:
(a) a polypeptide according to the present invention substantially as hereinbefore described;
(b) means for contacting either (i) a sample of body fluid obtained from said subject, or (ii) lymphocytes isolated from a sample of body fluid obtained from said subject, with said polypeptide according to the present invention substantially as hereinbefore described (under conditions that allow interaction of tTG with autoantibodies or lymphocytes produced in response to tTG) so as to permit said polypeptide to interact with autoantibodies, or lymphocytes, produced in response to tTG, and present in, or isolated from, said sample; and
(c) means for monitoring the degree, or effect, of interaction of said polypeptide with either said autoantibodies, or said lymphocytes, produced in response to tTG and present in, or isolated from, said sample, thereby providing an indication of the presence of said autoantibodies, or lymphocytes, in said sample or isolated from said sample.

Substantially as described above, a kit according to the present invention is suitable for screening for autoantibodies or lymphocytes produced in response to tTG in a sample of body fluid obtained from a subject. A kit according to the present invention can, however, be particularly adapted for use in screening for autoantibodies produced in response to tTG in a sample of body fluid obtained from a subject substantially as hereinafter described in greater detail.

There is in particular provided by the present invention, therefore, a kit for screening for autoantibodies produced in response to tTG in a sample of body fluid obtained from a subject (in particular a human) suspected of suffering from, susceptible to, having or recovering from autoimmune disease associated with an immune reaction to tTG, said kit comprising:
(a) a polypeptide according to the present invention substantially as hereinbefore described;
(b) means for contacting a sample of body fluid obtained from said subject with said polypeptide according to the present invention substantially as hereinbefore described (under conditions that allow interaction of tTG with autoantibodies produced in response to tTG) so as to permit said polypeptide to interact with autoantibodies produced in response to tTG and present in said sample; and
(c) means for monitoring the degree of interaction of said polypeptide with said autoantibodies produced in response to tTG and present in said sample, thereby providing an indication of the presence of said autoantibodies in said sample.

A kit according to the present invention may typically further comprise control means, such as means for providing a sample of body fluid from a normal subject, in other words a subject known to be without autoimmune disease associated with an immune reaction to tTG.

A kit for screening for autoantibodies to tTG according to the present invention may comprise means for directly monitoring interaction of (i) autoantibodies to tTG present in the sample of body fluid from the subject and (ii) a polypeptide, as provided by the present invention substantially as hereinbefore described, typically comprising non-competitive sandwich type assay means known in the art.

Typically, in a kit according to the present invention comprising non-competitive assay means, means are provided for monitoring the degree of interaction of (i) autoantibodies to tTG present in the sample and (ii) a polypeptide according to the present invention substantially as hereinbefore described, and can comprise labelling means provided either to a polypeptide according to the present invention substantially as hereinbefore described, or to a binding partner for autoantibodies to tTG, either of which would enable monitoring of the above described interaction. For example, a kit according to the present invention may comprise means for directly or indirectly labelling a polypeptide according to the present invention substantially as hereinbefore described; means for contacting the thus labelled polypeptide with a sample of body fluid being screened for tTG autoantibodies so as to provide a mixture thereof; a binding partner for autoantibodies to tTG (such as an anti-IgG reagent) present in the sample of body fluid; and means for adding the binding partner to the mixture so as to cause precipitation of any complexes of labelled polypeptide and tTG autoantibodies present in the mixture. Alternatively, it may be preferred that a kit according to the present invention further comprises a labelled binding partner for tTG autoantibodies (such as a labelled anti-IgG reagent, for example protein A or anti-human IgG, or labelled full length tTG or an epitope thereof) and means for adding the labelled binding partner to a mixture obtained by contacting (i) a polypeptide according to the present invention substantially as hereinbefore described immobilised to a support and (ii) a sample of body fluid being screened for autoantibodies to tTG.

It may alternatively be preferred that a kit for screening for autoantibodies to tTG in the sample of body fluid according to the present invention, comprises known competitive assay means. For example, a kit according to the present invention may further comprise at least one competitor capable of competing with autoantibodies to tTG in the interaction thereof with a polypeptide according to the present invention substantially as hereinbefore described.

Typically, a competitor as employed in a competitive assay kit according to the present invention may comprise one or more antibodies, which may be natural or partly or wholly synthetically produced. A competitor as employed in the present invention may alternatively comprise any other protein having a binding domain or region which is capable of competing with autoantibodies to tTG in the interaction thereof with a polypeptide according to the present invention substantially as hereinbefore described. Preferably, however, a competitor as employed in the present invention comprises a monoclonal or polyclonal antibody (especially a monoclonal antibody), capable of competing with tTG autoantibodies in the interaction thereof with a polypeptide according to the present invention substantially as hereinbefore described.

Typically, therefore, a competitive assay kit according to the present invention may further comprise at least one competitor, such as a monoclonal or polyclonal antibody, whereby a polypeptide according to the present invention substantially as hereinbefore described can interact with either a competitor, such as a monoclonal or polyclonal antibody, or autoantibodies to tTG present in a sample of body fluid being screened.

Typically monitoring means in a competitive assay kit according to the present invention comprise means for comparing:
(i) interaction of a polypeptide according to the present invention substantially as hereinbefore described and one or more competitors substantially as hereinbefore described (typically a monoclonal or polyclonal antibody), in the absence of said sample of body fluid being screened (that is a suspected disease sample), optionally in the presence of a sample of body fluid from a normal subject, typically a subject known to be without autoimmune disease associated with an immune reaction to tTG; with
(ii) interaction of a polypeptide according to the present invention substantially as hereinbefore described and one or more competitors substantially as hereinbefore described (typically a monoclonal or polyclonal antibody), in the presence of said sample of body fluid being screened.

Typically, the comparison involves observing a decrease in interaction of a polypeptide according to the present invention substantially as hereinbefore described and the competitor in (ii) compared to (i) so as to provide an indication of the presence of autoantibodies to tTG in said sample. Typically, the decrease in interaction can be observed by directly or indirectly labelling the competitor and monitoring any change in the interaction of the thus labelled competitor with a polypeptide according to the present invention substantially as hereinbefore described in the absence and in the presence of a sample of body fluid being screened for autoantibodies to tTG. Suitably a polypeptide according to the present invention substantially as hereinbefore described may be immobilised to facilitate the above mentioned monitoring.

Alternatively, there is also provided by the present invention a kit for screening for autoantibodies to tTG in a sample of body fluid obtained from a subject (in particular a human) suspected of suffering from, susceptible to, having or recovering from autoimmune disease associated with an immune reaction to tTG, said kit comprising:
(a) full length tTG (typically recombinantly obtained full length tTG);
(b) at least one competitor capable of competing with autoantibodies to tTG in the interaction thereof with a polypeptide according to the present invention substantially as hereinbefore described,

(c) means for contacting said sample of body fluid from said subject, said full length tTG and said competitor (under conditions that allow interaction of tTG with autoantibodies to tTG), so as to permit said full length tTG to interact with either autoantibodies to tTG present in said sample, or said competitor; and
(d) means for monitoring the interaction of said full length tTG with said autoantibodies present in said sample, thereby providing an indication of the presence of said autoantibodies to tTG in said sample.

The full length tTG can typically be of human, guinea pig, bovine, mouse or rat origin, preferably human, and more preferably human recombinantly obtained full length tTG. A competitor for use in such an assay kit typically comprises a monoclonal or polyclonal antibody (preferably monoclonal) substantially as hereinbefore described.

Suitably a detectable label that can be employed in a kit according to the present invention can be selected from the group consisting of enzymic labels, isotopic labels, chemiluminescent labels, fluorescent labels, dyes and the like.

In the case where an isotopic label (such as ¹²⁵I, ¹⁴C, ³H or ³⁵S) is employed, monitoring means may therefore comprise means for measuring radioactivity dependent on binding of a polypeptide according to the present invention substantially as hereinbefore described. Radioactivity is generally measured using a gamma counter, or liquid scintillation counter.

In the case of a kit for screening for lymphocytes according to the present invention, it is generally preferred that means are provided for initially isolating lymphocytes from a sample of body fluid from a subject, using techniques well known to one of ordinary skill in the art, and means are also provided for contacting a polypeptide according to the present invention to the present invention with such isolated lymphocytes so as to stimulate proliferation of the latter by the former. Means (again known to one of ordinary skill in the art) for monitoring the effect of interaction of a polypeptide according to the present invention and such proliferating lymphocytes, are also provided in such a kit according to the present invention.

There is still further provided by the present invention an antibody produced in response to one or more epitope regions of tTG, which epitope region comprises part or all of the primary structural conformation of amino acids 1 to 89 of tTG, or part or all of the primary structural conformation of amino acids 401 to 494 or 401 to 491 of tTG, or one or more fragments of an antibody (such as Fab fragments) produced in response to one or more epitope regions of tTG, which epitope region comprises part or all of the primary structural conformation of amino acids 1 to 89 of tTG, or part or all of the primary structural conformation of amino acids 401 to 494 or 401 to 491 of tTG.

An antibody according to the present invention or fragment thereof can interact with a polypeptide according to the present invention substantially as hereinbefore described and preferably is obtainable by, or is obtained by, techniques described in the Examples. Suitably, an antibody provided by the present invention can be monoclonal (preferred), recombinant or polyclonal. Typically an antibody, such as a monoclonal antibody, as provided by the present invention is in substantially purified form.

More specifically, a monoclonal antibody as provided by the present invention can comprise a monoclonal antibody Mab A3-6B5, or a monoclonal antibody Mab A5-4E6, or one or more active fragments thereof (such as Fab fragments), as described in the Examples.

The present invention further provides a host cell containing an antibody substantially as described above and also a hybridoma capable of secreting a monoclonal antibody substantially as hereinbefore described.

There is still further provided by the present invention an antibody substantially as hereinbefore described, or one or more fragments of an antibody (such as Fab fragments) substantially as hereinbefore described, for use in diagnosis or therapy, in particular diagnosis or therapy of autoimmune disease associated with an immune reaction to tTG substantially as hereinbefore described. Particularly the autoimmune disease can be coeliac disease (although other autoimmune diseases substantially as hereinafter described in greater detail can be diagnosed or treated by an antibody according to the present invention) and the present invention also provides an antibody substantially as hereinbefore described for use in the manufacture of a medicament for the treatment of coeliac disease.

There is still further provided by the present invention a polypeptide according to the present invention substantially as hereinbefore described, for use in diagnosis or therapy, in particular diagnosis or therapy of autoimmune disease associated with an immune reaction to tTG substantially as hereinbefore described.

In particular, autoimmune disease associated with an immune reaction to tTG as described herein comprises autoimmune disease arising due to gluten sensitive enteropathy in a subject, in particular a human subject, such as coeliac disease. Other autoimmune diseases associated with an immune reaction to tTG that can be diagnosed or treated by the present invention include any of the following—dermatitis herpetiformis, Addison's disease, A1 (A1-autoimmune) haemolytic anaemia, A1 thrombocytopenic purpura, A1 thyroid diseases, A1 hyperthyroidism (Graves' disease), A1 hypothyroidism (including Hashimoto's thyroiditis), pernicious anaemia, myasthenia gravis, primary biliary cirrhosis, rheumatoid arthritis, Sjogren's syndrome, SLE, type 1 (insulin-dependent) diabetes mellitus, diabetes mellitus of other types, Wegener granulomatosis, and vitiligo.

Substantially as herein described autoimmune disease associated with an immune reaction to tTG can be screened for or diagnosed in a subject suspected of suffering from, susceptible to, having or recovering from autoimmune disease associated with an immune reaction to tTG. The patient group that can be classed as susceptible to an autoimmune disease associated with an immune reaction to tTG can typically include relatives of subjects previously diagnosed as having an autoimmune disease associated with an immune reaction to tTG, or subjects previously diagnosed with a primary or first autoimmune disease associated with an immune reaction to tTG and as such could be considered to be at risk of or prone to be suffering from a secondary or second autoimmune disease associated with an immune reaction to tTG. For example, a subject known to be suffering from type 1 diabetes mellitus could be considered to be susceptible to suffering from coeliac disease.

It will be appreciated from the foregoing description that the present invention provides assay methods and kits for detecting autoantibodies (in particular) or lymphocytes produced in response to tTG in a sample of body fluid substantially as hereinbefore described. The detection of such autoantibodies and/or lymphocytes produced in response to tTG in the sample of body fluid (or at least the level of such autoantibodies and/or lymphocytes in the sample) is indicative of the presence of autoimmune disease associated with an immune reaction to tTG in the subject from which the sample was obtained and can, therefore, enable the diagnosis of the likely onset or presence of autoimmune disease associated with an immune reaction to tTG.

There is, therefore, further provided by the present invention a method of diagnosing the likely onset or presence of autoimmune disease associated with an immune reaction to tTG in a subject (in particular a human) suspected of suffering from, susceptible to, having or recovering from, autoimmune disease associated with an immune reaction to tTG, the method comprising detecting autoantibodies or lymphocytes produced in response to tTG in a sample of body fluid from the subject substantially as hereinbefore described, and whereby the detected autoantibodies and/or lymphocytes can provide a diagnosis of the likely onset or presence of autoimmune disease associated with an immune reaction to tTG in the subject.

There is still further provided by the present invention a method of delaying or preventing the onset of autoimmune disease associated with an immune reaction to tTG in an animal subject (in particular a human subject) suspected of suffering from, susceptible to or recovering from autoimmune disease associated with an immune reaction to tTG, which method comprises initially detecting autoantibodies or lymphocytes indicative of the onset or presence of autoimmune disease associated with an immune reaction to tTG in a sample of body fluid obtained from the subject substantially as hereinbefore described, thereby providing a diagnosis of the likely onset of autoimmune disease associated with an immune reaction to tTG in the subject, and thereafter therapeutically treating the subject so as to delay the onset and/or prevent autoimmune disease associated with an immune reaction to tTG. Typically, such therapeutic treatment can, in the case of gluten sensitive enteropathy giving rise to coeliac disease, comprise compliance with a gluten-free or substantially gluten free diet.

The present invention can further provide a method of monitoring dietary compliance by a subject (in particular a human subject) having, suspected of suffering from, susceptible to or recovering from gluten sensitive enteropathy, which method comprises screening for autoantibodies or lymphocytes produced in response to tTG in a sample of body fluid obtained from the subject substantially as hereinbefore described, and whereby the detection of such autoantibodies and/or lymphocytes produced in response to tTG in the sample of body fluid (or at least the level of such autoantibodies and/or lymphocytes in the sample) provides an indication as to the dietary compliance of the subject to a gluten-free or substantially gluten free diet.

A polypeptide according to the present invention substantially as hereinbefore described is particularly suitable for use in the therapeutic treatment of autoimmune disease associated with an immune reaction to tTG. For example, tolerance can be achieved by administering a polypeptide according to the present invention substantially as hereinbefore described to a subject (in particular a human subject) suspected of suffering from, susceptible to, having or recovering from autoimmune disease associated with an immune reaction to tTG.

There is, therefore, further provided by the present invention a pharmaceutical composition comprising a polypeptide according to the present invention substantially as hereinbefore described, together with a pharmaceutically acceptable carrier, diluent or excipient therefor, wherein the polypeptide can interact with autoantibodies and/or lymphocytes produced in response to tTG.

The present invention further provides a polypeptide according to the present invention substantially as hereinbefore described for use in the manufacture of a medicament for the treatment of coeliac disease.

Compositions or medicaments according to the present invention should contain a therapeutic or prophylactic amount of at least one polypeptide according to the present invention in a pharmaceutically-acceptable carrier. The pharmaceutical carrier can be any compatible, non-toxic substance suitable for delivery of the polypeptides to the patient. Sterile water, alcohol, fats, waxes, and inert solids may be used as the carrier. Pharmaceutically-acceptable adjuvants, buffering agents, dispersing agents and the like, may also be incorporated into the pharmaceutical compositions. Such compositions can contain a single polypeptide or may contain two or more polypeptides according to the present invention.

It may be desirable to couple a polypeptide according to the present invention to immunoglobulins, e.g. IgG, or to lymphoid cells from the patient being treated in order to promote tolerance. Such an approach is described in Bradley-Mullen, Activation of Distinct Subsets of T Suppressor Cells with Type III Pneumococcal Polysaccharide Coupled to Syngeneic Spleen Cells, in: IMMUNOLOGICAL TOLERANCE TO SELF AND NON-SELF, Buttisto et al., eds., Annals N.Y. Acad. Sci. Vol. 392, pp 156-166, 1982. Alternatively, the polypeptides may be modified to maintain or enhance binding to the MHC while reducing or eliminating binding to the associated T-cell receptor. In this way, the modified polypeptides may compete with natural tTG to inhibit helper T-cell activation and thus inhibit the immune response. In all cases, care should be taken that administration of the pharmaceutical compositions of the present invention ameliorate but do not potentiate the autoimmune response.

Pharmaceutical compositions according to the present invention are useful for parenteral administration. Preferably, the compositions will be administered parenterally, i.e. subcutaneously, intramuscularly, or intravenously. Thus, the invention provides compositions for parenteral administration to a patient, where the compositions comprise a solution or dispersion of the polypeptides in an acceptable carrier, as described above. The concentration of the polypeptides in the pharmaceutical composition can vary widely, i.e. from less than about 0.1% by weight, usually being at least about 1% by weight to as much as 20% by weight or more. Typical pharmaceutical compositions for intramuscular injection would be made up to contain, for example, 1 ml of sterile buffered water and 1 to 100 μg of a purified polypeptide of the present invention. A typical composition for intravenous infusion could be made up to contain 100 to 500 ml of sterile Ringer's solution and 100 to 500 mg of a purified polypeptide of the present invention. Actual methods for preparing parenterally administrable compositions are well known in the art and described in more detail in various sources, including, for example, Remington's Pharmaceutical Science, 15^{th} Edition, Mack Publishing Company, Easton, Pa. (1980).

In addition to using a polypeptide according to the present invention directly in pharmaceutical compositions, it is also possible to use a polypeptide according to the present invention to enhance tolerance to tTG in a subject suspected of suffering from, susceptible to, having or recovering from autoimmune disease associated with an immune reaction to tTG, employing the following principles. More particularly, peripheral blood lymphocytes can be collected from the subject in a conventional manner and stimulated by exposure to a polypeptide according to the present invention, as defined above. Usually, other mitogens and growth enhancers will be present, e.g., phytohemagglutinin, interleukin 2, and the like. Proliferating T-helper cells may be isolated and cloned, also under the stimulation of a polypeptide according to the present invention. Clones which continue to proliferate may then be used to prepare therapeutic compositions for the subject. The cloned T-cells may be attenuated, e.g. by exposure to radiation, and administered to the subject in order to induce tolerance. Alternatively, the T-cell receptor or portions thereof may be isolated by conventional protein purification methods from the cloned T-cells and administered to the individual. Such immunotherapy methods are described generally in Sinha et al. (1990) Science 248:1380-1388.

In some cases, after a T-helper cell has been cloned as described above, it may be possible to develop therapeutic peptides from the T-cell receptor, where the peptides would be beneficial for treating a patient population suspected of suffering from, susceptible to, having or recovering from autoimmune disease associated with an immune reaction to tTG. In such cases, the T-cell receptor gene may be isolated and cloned by conventional techniques and peptides based on the receptor produced by recombinant techniques as described above. The recombinantly-produced peptides may then be incorporated in pharmaceutical compositions as described above.

There is also provided by the present invention a method of cloning lymphocytes produced in response to tTG, which method comprises:
providing a source of lymphocytes;
contacting the lymphocytes with a polypeptide according to the present invention substantially as hereinbefore described, so as to effect proliferation of said lymphocytes; and
isolating and cloning the proliferating lymphocytes.

The present invention also provides the use of cloned lymphocytes prepared as above, in the therapeutic treatment of autoimmune disease associated with an immune reaction to tTG. There is provided, therefore, a pharmaceutical composition comprising cloned lymphocytes prepared as above, together with a pharmaceutically acceptable carrier, diluent or excipient therefor and the use of such cloned lymphocytes in the manufacture of a medicament for the treatment of autoimmune disease associated with an immune reaction to tTG, in particular coeliac disease.

There is also provided by the present invention one or more therapeutic agents identified as providing a therapeutic effect by interaction with amino acids 1 to 89 of tTG, and/or amino acids 401 to 494 of tTG, or amino acids 401 to 491 of tTG, and the present invention further provides one or more therapeutic agents for use in therapeutically interacting with amino acids 1 to 89 of tTG, and/or amino acids 401 to 494 of tTG or amino acids 401 to 491 of Ttg and as such for use in the therapeutic treatment of an autoimmune disease associated with an immune reaction to tTG.

There is, therefore, still further provided by the present invention a method of treating autoimmune disease associated with an immune reaction to tTG in a subject, which method comprises initially detecting autoantibodies or lymphocytes produced in response to tTG in a sample of body fluid obtained from the subject substantially as hereinbefore described, thereby providing a diagnosis of autoimmune disease in the subject, and administering to the subject a therapeutically effective amount of at least one therapeutic agent effective in the treatment of such autoimmune disease, such as a polypeptide according to the present invention substantially as hereinbefore described.

The present invention also provides a method of treating autoimmune disease associated with an immune reaction to tTG in a subject (in particular a human subject), which method comprises administering to the subject a therapeutically effective amount of a therapeutic agent identified as providing a therapeutic effect by interaction with amino acids 1 to 89 of tTG, and/or amino acids 401 to 494 of tTG, or amino acids 401 to 491 of tTG.

The amount of therapeutic agent administered will depend on the specific autoimmune disease state being treated, possibly the age of the patient and will ultimately be at the discretion of an attendant physician.

There is still further provided by the present invention, in combination, a kit substantially as hereinbefore described, together with a therapeutically effective amount of at least one therapeutic agent effective in the treatment of autoimmune disease associated with an immune reaction to tTG substantially as hereinbefore described.

Substantially as hereinbefore described, the sample of body fluid being screened by the present invention will typically comprise blood samples or other fluid blood fractions, such as in particular serum samples or plasma samples, but the sample may in principle be another biological fluid, such as saliva or urine or solubilised tissue extracts.

The present invention will now be illustrated with reference to the accompanying Figures and Examples, which do not limit the invention in any way.

FIG. 1 shows results of SDS PAGE (9%) analysis of purified recombinant human tTG for use in the method and kit according to the invention.

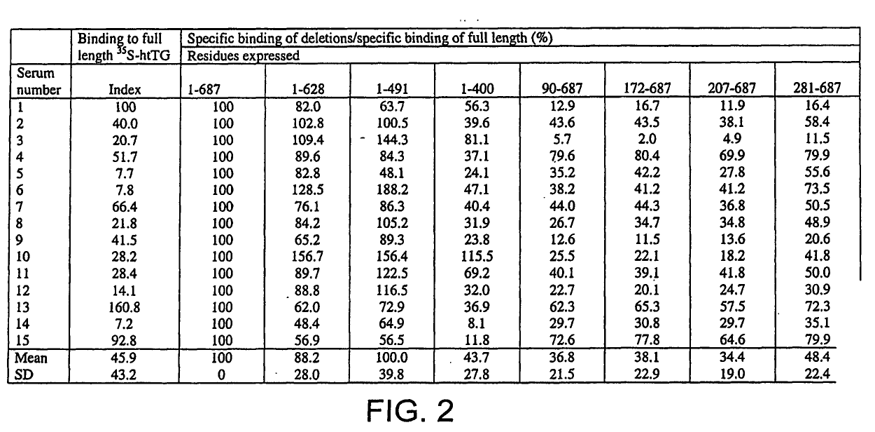

FIG. 2 is a table showing binding of autoantibodies to tTG in sera from coeliac patients with full length and modified ³⁵S-tTG proteins.

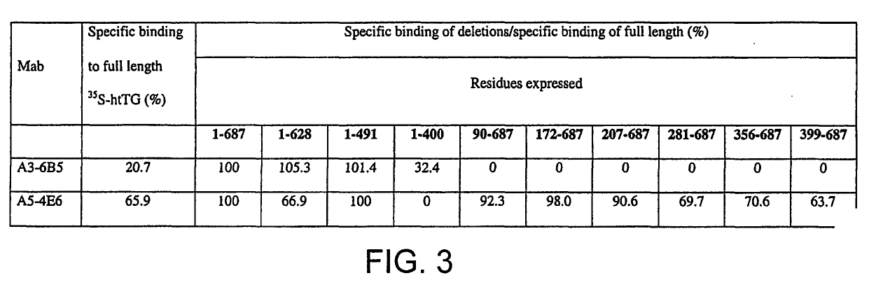

FIG. 3 is a table showing monoclonal antibody (namely monoclonal antibodies A3-6B5 and A5-4E6) binding to full length and modified ³⁵S-tTG proteins.

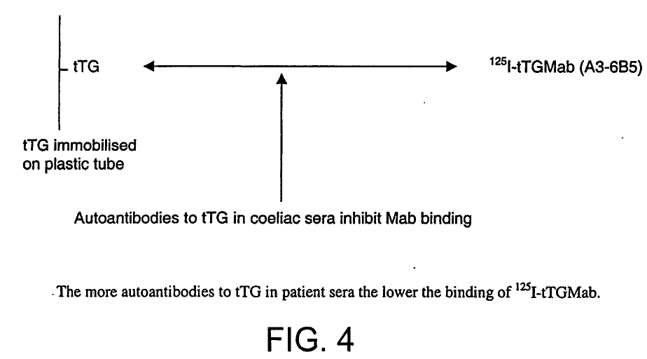

FIG. 4 shows a schematic example of the method of the present invention when an antibody is employed.

FIG. 5 is a graph showing binding (absorbance at 450 nm in the vertical axis) of tTG with autoantibodies to tTG in different patients sera (horizontal axis). The binding is measured in the presence or absence of mouse tTG monoclonal antibody A3-6B5.

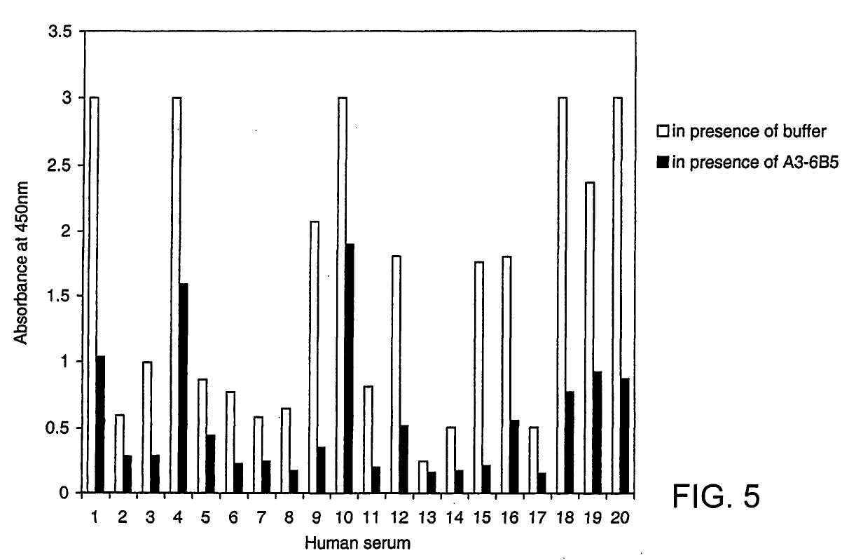

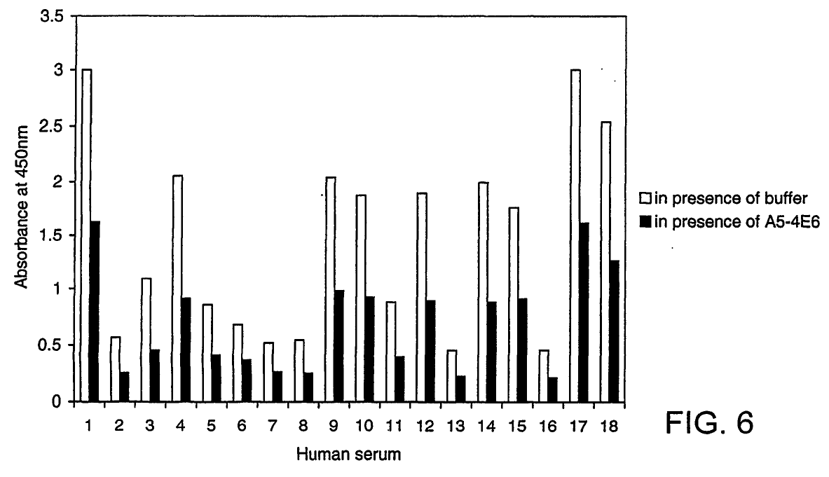

FIG. 6 is a graph showing binding (absorbance at 450 nm in the vertical axis) of tTG with autoantibodies to tTG in different patients sera (horizontal axis). The binding is measured in the presence or absence of mouse tTG monoclonal antibody A5-4E6.

FIG. 7 depicts cDNA derived from human tTG, encoding amino acids 1 to 89 of human tTG.

FIG. 8 depicts amino acids 1 to 89 of human tTG.

FIG. 9 depicts cDNA derived from guinea pig tTG, encoding amino acids 1 to 89 of guinea pig tTG.

FIG. 10 depicts amino acids 1 to 89 of guinea pig tTG.

FIG. 11 depicts CDNA derived from bovine tTG, encoding amino acids 1 to 89 of bovine tTG.

FIG. 12 depicts amino acids 1 to 89 of bovine tTG.

FIG. 13 depicts cDNA derived from mouse tTG, encoding amino acids 1 to 89 of mouse tTG.

FIG. 14 depicts amino acids 1 to 89 of mouse tTG.

FIG. 15 depicts cDNA derived from rat tTG, encoding amino acids 1 to 89 of rat tTG.

FIG. 16 depicts amino acids 1 to 89 of rat tTG.

FIG. 17 depicts cDNA derived from human tTG, encoding amino acids 401 to 491 of human tTG.

FIG. 18 depicts amino acids 401 to 491 of human tTG.

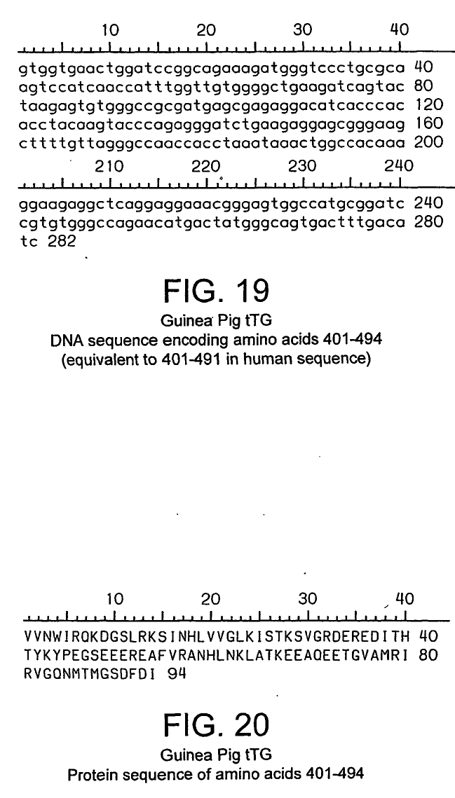

FIG. 19 depicts cDNA derived from guinea pig tTG, encoding amino acids 401 to 494 of guinea pig tTG.

FIG. 20 depicts amino acids 401 to 494 of guinea pig tTG.

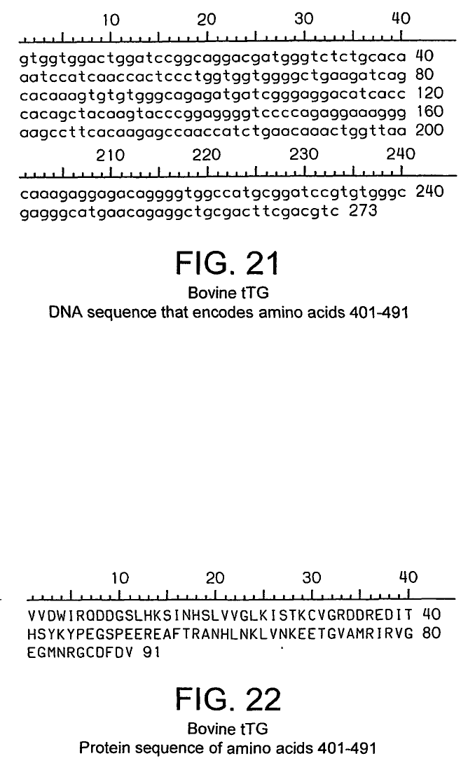

FIG. 21 depicts cDNA derived from bovine tTG, encoding amino acids 401 to 491 of bovine tTG.

FIG. 22 depicts amino acids 401 to 491 of bovine tTG.

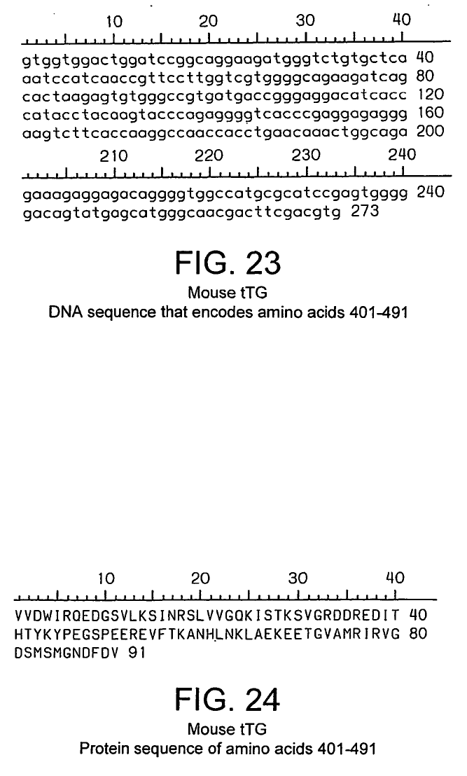

FIG. 23 depicts cDNA derived from mouse tTG, encoding amino acids 401 to 491 of mouse tTG.

FIG. 24 depicts amino acids 401 to 491 of mouse tTG.

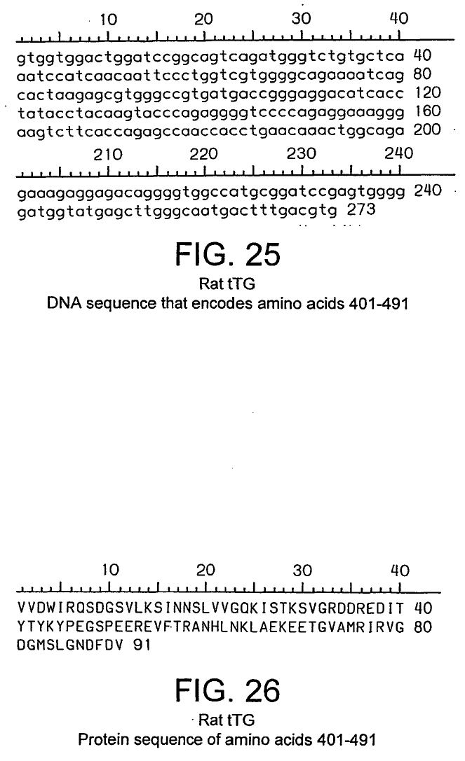

FIG. 25 depicts cDNA derived from rat tTG, encoding amino acids 401 to 491 of rat tTG.

FIG. 26 depicts amino acids 401 to 491 of rat tTG.

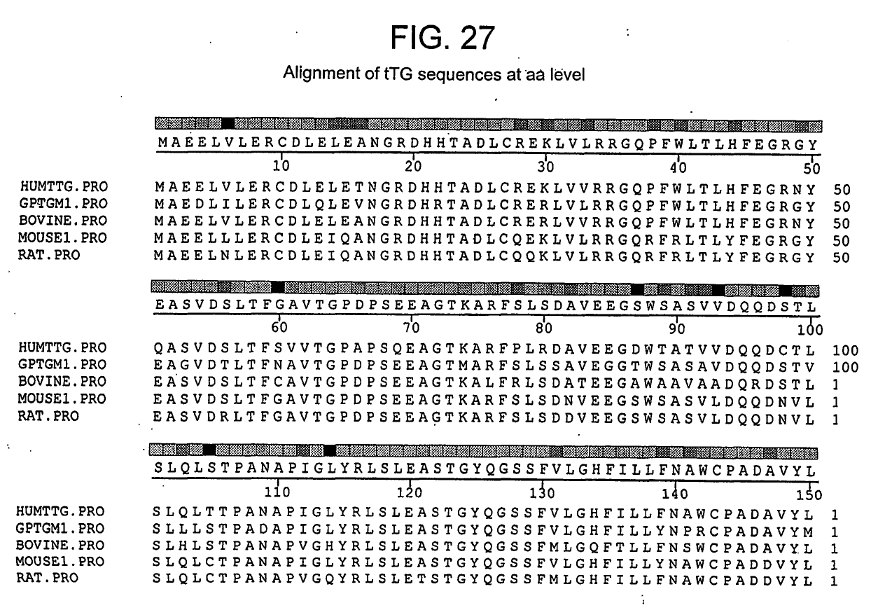

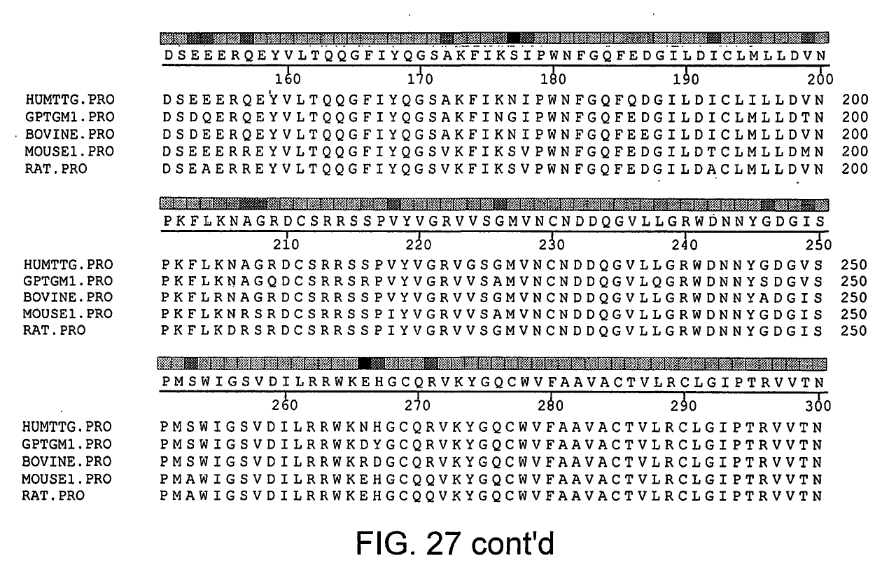

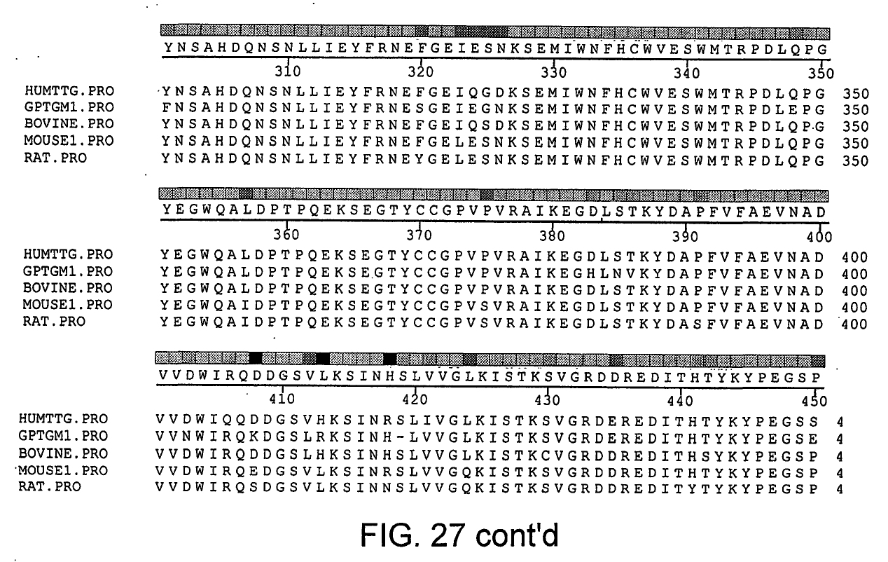

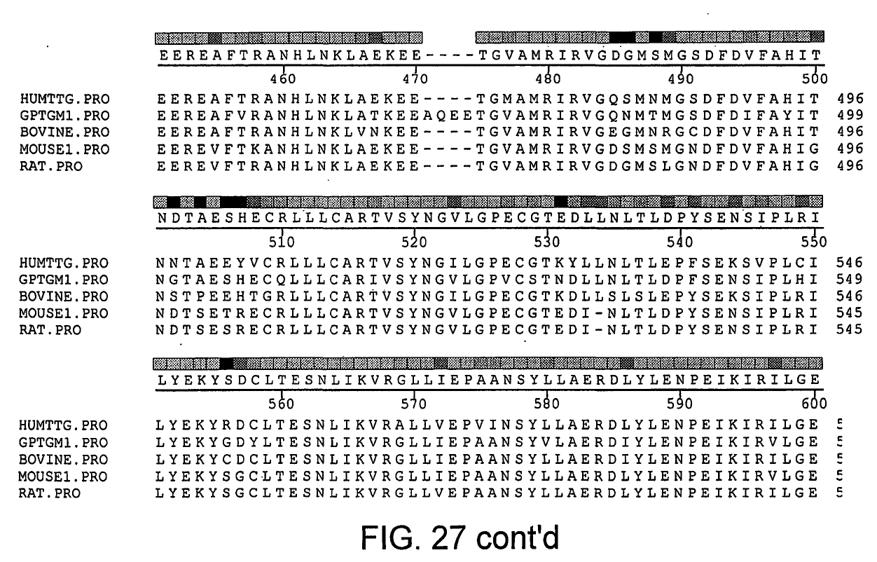

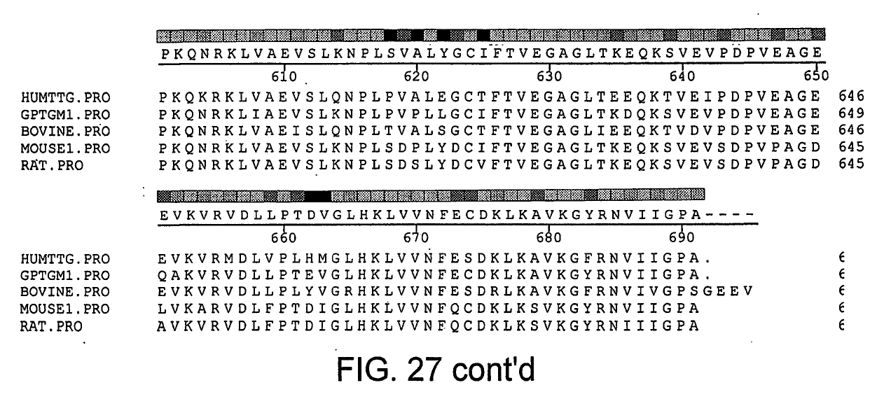

FIG. 27 depicts the alignment of tTG sequences (human, guinea pig, bovine, mouse and rat) at the amino acid level.

Further illustration of the present invention is given in the following detailed worked Examples.

### EXAMPLES

### Identification of Regions of tTG which are Recognised by tTG Autoantibodies

Full length human tTG cDNA (endothelial cell isoform) was cloned into pTZ18R (Pharmacia) under control of the SP6 promoter. This construct was used to produce a reference full length tTG protein. Various in-frame deletions and truncations of the tTG gene were carried out using either naturally occurring restriction endonuclease sites or PCR mediated modifications to the sequence. The modified tTG constructs were cloned into pTZ18R (as above).

Full length and modified tTG proteins were expressed in an in vitro transcription/translation rabbit reticulocyte system (Promega) incorporating ³⁵S-methionine (Amersham Pharmacia Biotech) and purified as described before (Colls J, et al. Clin. Chem. 1995; 41/3:375-380). Table 1 shows the vectors used and the corresponding tTG amino acid residues expressed following the in vitro transcription/translation described above.

TABLE 1

| | | |
|---|---|---|
| | | Non-expressed |
| Vector Name | Expressed residues | (deleted) residues |

| | | | |
|---|---|---|---|
| pTGM19 | 1-687 | (full length) | None |
| pTGM25 | 1-628 | | 629-687 |
| pTGM22 | 1-491 | | 492-687 |
| pTGM24 | 1-400 | | 401-687 |
| pTGM26 | 90-687 | | 1-89 |
| pTGM27 | 172-687 | | 1-171 |
| pTGM28 | 207-687 | | 1-206 |
| pTGM29 | 281-687 | | 1-280 |
| pTGM30 | 356-687 | | 1-355 |
| pTGM31 | 399-687 | | 1-398 |

Binding of tTG autoantibodies to the purified full length and modified tTG proteins was analysed using an immunoprecipitation assay (Nakachi K, et al Clin Chim Acta, Volume 304, pages 75 to 84, 2002). Briefly, 10 μL of undiluted serum (in duplicate) was incubated with 50 μL of ³⁵S-tTG (approx 15,000 dpm diluted in 10 mmol/L Tris HCl pH 7.6, 150 mmol/L NaCl, 2 mmol/L EDTA, 0.1% Tween 20, 10 g/L bovine serum albumin, 0.2 g/L NaN₃; Assay Buffer) in Millipore filtration plates overnight at 4° C. Anti-human IgA agarose (50 μl) was then added and the plates incubated for 4 hours at 4° C. After incubation the reaction mixtures in the wells were washed 3 times. The wells were then punched out into vials containing 1 mL of scintillation liquid and counted in a beta-counter. FIG. 2 shows binding of tTG autoantibodies with different modified tTG proteins. The results are expressed as % binding relative to binding to the full length tTG reference protein.

Serum Samples

Serum from 34 patients with coeliac disease were used in the study. The diagnosis was based on typical clinical symptoms and was confirmed by positive duodenal biopsy. The sera tested positive for endomysial antibody and autoantibodies to tTG by standard ELISA. In addition sera from healthy blood donors (n=10) negative for autoantibodies to tTG were used.

Production of Mouse Monoclonal Antibodies

Both guinea pig tTG (Sigma, Poole, UK) and recombinant human tTG (rhtTG) were used as antigens to immunise mice. Monoclonal antibodies (Mabs) were cloned using standard techniques. Selected tTG Mabs were grown in culture and purified by protein A affinity chromatography. The location of the binding sites of the mouse monoclonal antibodies was determined by an immunoprecipitation assay using the modified ³⁵S-labelled tTG proteins as described above, except that anti-mouse IgG was added for precipitation. When required, purified IgG was labelled with ¹²⁵I using the chloramine T method.

Analysis of the mouse Mabs by immunoprecipitation with modified ³⁵S-labelled tTG proteins indicated that the epitope for A3-6B5 Mab was contained within aa 1-89 of the human tTG protein and the epitope for A5-4E6 Mab was contained within aa 401-491 of the human tTG protein (FIG. 3).

Inhibition of Autoantibodies to tTG Binding with tTG by Mouse tTG Mabs

The inhibition of autoantibodies to tTG binding with tTG by mouse tTG Mabs was investigated in a modified version of an ELISA described in Nakachi K, et al. Clin Chim Acta (Volume 304, pages 75 to 84, 2002). To streptavidin coated plate wells 50 μL of tTG-biotin was added plus 10μL of Mab (200 μ/ml) or Assay Buffer and incubated for 30 mins. 40 μL of patients' sera (typically diluted 1:100) was then added and incubated for 30 mins. After this step all wells were aspirated, washed four times and then 100 μL of anti-human IgA-horseradish peroxidase (HRP) was added and incubated for 30 mins. After aspiration and four washes, 100 μL of tetramethyl benzidine (TMB) solution was added and the plate was incubated for 15 mins in the dark. 50 μL of stop solution was then added and absorbance read at 450 nm in a plate reader. All steps were performed at room temperature, all incubations except the final TMB incubation were done with 200 rpm shaking. tTG-biotin, Mabs and human sera were diluted in Assay Buffer. The results are shown in FIG. 5 which shows binding of tTG with autoantibodies to tTG in different patients sera in the presence or absence of mouse tTG monoclonal antibody A3-6B5 and FIG. 6 which shows binding of tTG with autoantibodies to tTG in different patients sera in the presence or absence of mouse tTG monoclonal antibody A5-4E6.

Preparation of Purified Recombinant Human tTG

Recombinant human tTG was expressed in the yeast Saccharomyces cerevisiae strain c13ABYS86 using an expression system and culture techniques described previously (Powell M, et al. Clin Chim Acta 1996; 256: 175-188). Recombinant human tTG extracted from the yeast was partially purified by anion exchange chromatography and preparations of tTG of greater than 95% purity were obtained by affinity chromatography using mouse tTG Mab (A5-6C7) coupled to CNBr Sepharose. The purity of the purified tTG was assessed by SDS PAGE analysis on a 9% acrylamide gel followed by staining with Coomassie Blue as shown in FIG. 1.

Assay for Screening a Sample of Body Fluid for Autoantibodies to tTG Based on the Inhibition of ¹²⁵I-Mab Binding to Recombinant Human tTG

An assay was set up based on the principles of the schematic example shown in FIG. 4. 4 mL Nunc Maxisorp Startubes (Life Technologies) were coated overnight at 4° C. with 250 μL of 10 μg/mL recombinant human tTG (rhtTG). Following aspiration and three washes with 500 μL Assay Buffer, tubes were incubated for 30 minutes at room temperature with 500 μL of Post-Coat Buffer containing bovine serum albumin. After two washes with Assay Buffer, tubes were incubated with 100 μL of serum sample diluted 1:5 in Assay Buffer for 60 minutes at room temperature on a shaker. Following aspiration and three washes with 500 μL of Assay Buffer, tubes were incubated on a shaker for 30 minutes at room temperature with 100 μL of ¹²⁵I-labelled tTG Mab A3-6B5 (30,000 cpm; diluted in Assay Buffer). After a final aspiration and 3 washes with 500 μL of Assay Buffer, tubes were counted for 1 minute in a gamma counter.

The results, measured in counts per minute (cpm), were used to calculate the % inhibition of ¹²⁵I-Mab binding as follows:

$$\% inhibition = 100 - [ \frac{A}{B} \times 100 ]$$

wherein,
A=¹²⁵I-Mab (cpm) bound to rhtTG in the presence of test sera as a % of total cpm of material added to the tube; and
B=¹²⁵I-Mab (cpm) bound to rhtTG in the presence of healthy blood donor sera (mean of 2 or 3 sera) as a % of total cpm of material added to the tube.
   Results and Discussion

FIG. 2 showed that C-terminal deletions of amino acids (aa) 492-687 of the tTG protein had no or little effect on tTG autoantibody binding. More extensive C-terminal deletion (aa 401-687) resulted in lower tTG autoantibody binding in 14/15 sera studied. N-terminal deletion of aa 1-89 of the tTG protein resulted in lower tTG autoantibody binding in all sera studied.

Therefore, the studies with modified tTG proteins described herein showed that tTG autoantibody binding sites on tTG are heterogenous and are dependent on the aa sequences in the N-terminal and central part of the molecule. Furthermore, these studies showed that aa 1-89, and aa 401-491 of human tTG protein are important for binding of tTG autoantibodies present in the coeliac sera studied. (aa 401-491 of human tTG correspond to aa 401-494 of tTG in other species, such as guinea pig tTG.)

Analysis of mouse Mabs by immunoprecipitation with modified ³⁵S-labelled tTG proteins indicated that the epitope for A3-6B5 Mab was contained within aa 1-89 of the human tTG protein and the epitope for A5-4E6 Mab was contained within aa 401-491 of the human tTG protein (FIG. 3). Furthermore, these Mabs, namely A3-6B5 Mab and A5-4E6 Mab, inhibited binding of tTG autoantibody positive sera with tTG in ELISA (see FIGS. 5 and 6).

Identification of amino acid sequences of the tTG molecule important for binding of autoantibodies to tTG tested, and production of mouse Mabs reactive with these amino acid sequences, allowed the development of a new assay to screen for the presence of autoantibodies to tTG in a sample of body fluid as described above.

The results of the assay based on autoantibodies to tTG in test sera inhibiting ¹²⁵I-Mab A3-6B5 binding with recombinant human tTG are shown in Table 2.

TABLE 2
Results of assay based on the inhibition of
¹²⁵I-Mab binding to recombinant human tTG

| | % Inhibition of | tTG | ³⁵S rhtTG |
|---|---|---|---|
| Serum | ¹²⁵I-A3-6B5 Mab | Autoantibodies | Autoantibodies |
| Number | binding | ELISA u/mL | IPA Index |
| Coeliac | | | |
| 1 | 79 | 1000 | 100 |
| 2 | 35 | 110 | 52 |
| 3 | 91 | 42 | 7.7 |
| 4 | 49 | 150 | 21 |
| 5 | 89 | 1000 | 44 |
| 6 | 88 | 51 | 107 |
| 7 | 89 | 1000 | 122 |
| 8 | 42 | 102 | 107 |
| 9 | 48 | 45 | 3.3 |
| Healthy blood | | | |
| donors | | | |
| 1 | −11 | Neg | Neg |
| 2 | 13 | Neg | Neg |
| 3 | −1 | Neg | Neg |
| 4 | 3 | Neg | Neg |
| 5 | −3 | Neg | Neg |

Nine sera from coeliac patients which tested positive for tTG autoantibodies by immunoprecipitation assay (tTG autoantibodies index from 3.3-107) and ELISA (42-1000 u/mL) were analysed. All these sera inhibited binding of ¹²⁵I-Mab to rhtTG coated on the tube (35 to 91% inhibition).

In contrast, sera negative for autoantibodies to tTG only had a small effect on the binding of ¹²⁵I-Mab to rhtTG (−11 to 13% inhibition).

1. A DNA sequence encoding:
a polypeptide with which autoantibodies and/or lymphocytes produced in response to tTG can interact, under conditions that allow interaction of tTG with such autoantibodies or lymphocytes, and which comprises part or all of the primary structural conformation of one or more epitopes of tTG with which autoantibodies and/or lymphocytes produced in response to tTG can interact, under conditions that allow interaction of tTG with such autoantibodies or lymphocytes,:
which DNA sequence comprises:
(a) a DNA sequence as depicted in any of Seq. Ids 1, 3, 5, 7, 9, 11, 13, 15, 17, 19;
(b) a DNA sequence encoding a polypeptide comprising the primary structural conformation of amino acid numbers 1 to 89 of tTG, and/or the primary structural conformation of amino acid numbers 401 to 494 of tTG or the primary structural conformation of amino acid numbers 401 to 491 of tTG, or one or more active fragments of:
amino acid numbers 1 to 89 of tTG, and/or amino acid numbers 401 to 494 of tTG or amino acid numbers 401 to 491 of tTG, with which autoantibodies and/or lymphocytes produced in response to tTG can interact, under conditions that allow interaction of tTG with such autoantibodies or lymphocytes,
with the exception of human, guinea pig, mouse, bovine or rat full length tTG;
(c) a DNA sequence encoding a polypeptide comprising the primary structural conformation of amino acids as depicted in one or more of Seq. Ids 2, 4, 6, 8, 10, 12, 14, 16, 18 and 20, or one or more active fragments of amino acids as depicted in one or more of Seq. Ids 2, 4, 6, 8, 10, 12, 14, 16, 18 and 20, with which autoantibodies and/or lymphocytes produced in response to tTG can interact, under conditions that allow interaction of tTG with such autoantibodies or lymphocytes, with the exception of human, guinea pig, mouse, bovine or rat full length tTG;
(d) a DNA sequence differing from the DNA sequence of (a) in codon sequence due to the degeneracy of the genetic code;
(e) a DNA sequence comprising a fragment or an allelic variation of the DNA sequence of (a); and
(f) a DNA sequence which hybridizes to any of DNA sequences (a), (b), (c), (d) or (e) and encodes a polypeptide with which autoantibodies and/or lymphocytes produced in response to tTG can interact, under conditions that allow interaction of tTG with such autoantibodies or lymphocytes, and which comprises part or all of the primary structural conformation of one or more epitopes of tTG with which autoantibodies and/or lymphocytes produced in response to tTG can interact, under conditions that allow interaction of tTG with such autoantibodies or lymphocytes.

2. A DNA sequence encoding:
a polypeptide with which autoantibodies and/or lymphocytes produced in response to tTG can interact, under conditions that allow interaction of tTG with such autoantibodies or lymphocytes, and which comprises part or all of the primary structural conformation of one or more epitopes of tTG with which autoantibodies and/or lymphocytes produced in response to tTG can interact, under conditions that allow interaction of tTG with such autoantibodies or lymphocytes;
which DNA sequence comprises:
(a) a DNA sequence as depicted in any of Seq. Ids 1, 3, 5, 7 and 9;
(b) a DNA sequence encoding a polypeptide comprising the primary structural conformation of amino acid numbers 1 to 89 of tTG, or one or more active fragments of amino acid numbers 1 to 89 of tTG, with which autoantibodies and/or lymphocytes produced in response to tTG can interact, under conditions that allow interaction of tTG with such autoantibodies or lymphocytes, with the exception of human, guinea pig, mouse, bovine or rat full length tTG;
(c) a DNA sequence encoding a polypeptide comprising the primary structural conformation of amino acids as depicted in one or more of Seq. Ids 2, 4, 6, 8 and 10, or one or more active fragments of amino acids as depicted in one or more of Seq. Ids 2 4, 6, 8 and 10, with which autoantibodies and/or lymphocytes produced in response to tTG can interact, under conditions that allow interaction of tTG with such autoantibodies or lymphocytes, with the exception of human, guinea pig, mouse, bovine or rat full length tTG;
(d) a DNA sequence differing from the DNA sequence of (a) in codon sequence due to the degeneracy of the genetic code;
(e) a DNA sequence comprising a fragment or an allelic variation of the DNA sequence of (a); and
(f) a DNA sequence which hybridizes to any of DNA sequences (a), (b), (c), (d) or (e) and encodes a polypeptide with which autoantibodies and/or lymphocytes produced in response to tTG can interact, under conditions that allow interaction of tTG with such autoantibodies or lymphocytes, and which comprises part or all of the primary structural conformation of one or more epitopes of tTG with which autoantibodies and/or lymphocytes produced in response to tTG can interact, under conditions that allow interaction of tTG with such autoantibodies or lymphocytes.

3. A DNA sequence encoding:
a polypeptide with which autoantibodies and/or lymphocytes produced in response to tTG can interact, under conditions that allow interaction of tTG with such autoantibodies or lymphocytes, and which comprises part or all of the primary structural conformation of one or more epitopes of tTG with which autoantibodies and/or lymphocytes produced in response to tTG can interact, under conditions that allow interaction of tTG with such autoantibodies or lymphocytes;
which DNA sequence comprises:
(a) a DNA sequence as depicted in any of Seq. Ids 11, 13, 15, 17 or 19;
(b) a DNA sequence encoding a polypeptide comprising the primary structural conformation of amino acid numbers 401 to 494 of tTG or the primary structural conformation of amino acid numbers 401 to 491 of tTG, or one or more active fragments of:
amino acid numbers 401 to 494 of tTG or amino acid numbers 401 to 491 of tTG, with which autoantibodies and/or lymphocytes produced in response to tTG can interact, under conditions that allow interaction of tTG with such autoantibodies or lymphocytes,
with the exception of human, guinea pig, mouse, bovine or rat full length tTG;
(c) a DNA sequence encoding a polypeptide comprising the primary structural conformation of amino acids as depicted in one or more of Seq. Ids 12, 14, 16 and 18, or one or more active fragments of amino acids as depicted in one or more of Seq. Ids 12, 14, 16 and 18, with which autoantibodies and/or lymphocytes produced in response to tTG can interact, under conditions that allow interaction of tTG with such autoantibodies or lymphocytes, with the exception of human, guinea pig, mouse, bovine or rat full length tTG;
(d) a DNA sequence differing from the DNA sequence of (a) in codon sequence due to the degeneracy of the genetic code;
(e) a DNA sequence comprising a fragment or an allelic variation of the DNA sequence of (a); and
(f) a DNA sequence which hybridizes to any of DNA sequences (a), (b), (c), (d) or (e) and encodes a polypeptide with which autoantibodies and/or lymphocytes produced in response to tTG can interact, under conditions that allow interaction of tTG with such autoantibodies or lymphocytes, and which comprises part or all of the primary structural conformation of one or more epitopes of tTG with which autoantibodies and/or lymphocytes produced in response to tTG can interact, under conditions that allow interaction of tTG with such autoantibodies or lymphocytes.

4. A DNA sequence encoding:
a polypeptide with which autoantibodies produced in response to tTG can interact, under conditions that allow interaction of tTG with such autoantibodies, and which comprises part or all of the primary structural conformation of one or more epitopes of tTG with which autoantibodies produced in response to tTG can interact, under conditions that allow interaction of tTG with such autoantibodies;
which DNA sequence comprises:
(a) a DNA sequence as depicted in any of Seq. Ids 1, 3, 5, 7, 9, 11, 13, 15, 17, or 19;
(b) a DNA sequence encoding a polypeptide comprising the primary structural conformation of amino acid numbers 1 to 89 of tTG, and/or the primary structural conformation of amino acid numbers 401 to 494 of tTG or the primary structural conformation of amino acid numbers 401 to 491 of tTG, or one or more active fragments of:
amino acid numbers 1 to 89 of tTG, and/or amino acid numbers 401 to 494 of tTG or amino acid numbers 401 to 491 of tTG, with which autoantibodies produced in response to tTG can interact, under conditions that allow interaction of tTG with such autoantibodies,
with the exception of human, guinea pig, mouse, bovine or rat full length tTG;
(c) a DNA sequence encoding a polypeptide comprising the primary structural conformation of amino acids as depicted in one or more of Seq. Ids 2, 4, 6, 8, 10, 12, 14, 16, 18 and 20, or one or more active fragments of amino acids as depicted in one or more of Seq. Ids 2, 4, 6, 8, 10, 12, 14, 16, 18 and 20, with which autoantibodies produced in response to tTG can interact, under conditions that allow interaction of tTG with such autoantibodies, with the exception of human, guinea pig, mouse, bovine or rat full length tTG;
(d) a DNA sequence differing from the DNA sequence of (a) in codon sequence due to the degeneracy of the genetic code;
(e) a DNA sequence comprising a fragment or an allelic variation of the DNA sequence of (a); and
(f) a DNA sequence which hybridizes to any of DNA sequences (a), (b), (c), (d) or (e) and encodes a polypeptide with which autoantibodies produced in response to tTG can interact, under conditions that allow interaction of tTG with such autoantibodies, and which comprises part or all of the primary structural conformation of one or more epitopes of tTG with which autoantibodies produced in response to tTG can interact, under conditions that allow interaction of tTG with such autoantibodies.

5. A DNA sequence encoding:
a polypeptide with which autoantibodies produced in response to tTG can interact, under conditions that allow interaction of tTG with such autoantibodies, and which comprises part or all of the primary structural conformation of one or more epitopes of tTG with which autoantibodies produced in response to tTG can interact, under conditions that allow interaction of tTG with such autoantibodies;
which DNA sequence comprises:
(a) a DNA sequence as depicted in any of Seq. Ids 1, 3, 5, 7 or 9;
(b) a DNA sequence encoding a polypeptide comprising the primary structural conformation of amino acid numbers 1 to 89 of tTG, or one or more active fragments of amino acid numbers 1 to 89 of tTG, with which autoantibodies produced in response to tTG can interact, under conditions that allow interaction of tTG with such autoantibodies,
with the exception of human, guinea pig, mouse, bovine or rat full length tTG;
(c) a DNA sequence encoding a polypeptide comprising the primary structural conformation of amino acids as depicted in one or more of Seq. Ids 2, 4, 6, 8 and 10, or one or more active fragments of amino acids as depicted in one or more of Seq. Ids 2, 4, 6, 8 and 10, with which autoantibodies produced in response to tTG can interact, under conditions that allow interaction of tTG with such autoantibodies, with the exception of human, guinea pig, mouse, bovine or rat full length tTG;
(d) a DNA sequence differing from the DNA sequence of (a) in codon sequence due to the degeneracy of the genetic code;
(e) a DNA sequence comprising a fragment or an allelic variation of the DNA sequence of (a); and
(f) a DNA sequence which hybridizes to any of DNA sequences (a), (b), (c), (d) or (e) and encodes a polypeptide with which autoantibodies produced in response to tTG can interact, under conditions that allow interaction of tTG with such autoantibodies, and which comprises part or all of the primary structural conformation of one or more epitopes of tTG with which autoantibodies produced in response to tTG can interact, under conditions that allow interaction of tTG with such autoantibodies.

6. A DNA sequence encoding:
a polypeptide with which autoantibodies produced in response to tTG can interact, under conditions that allow interaction of tTG with such autoantibodies, and which comprises part or all of the primary structural conformation of one or more epitopes of tTG with which autoantibodies produced in response to tTG can interact, under conditions that allow interaction of tTG with such autoantibodies;
which DNA sequence comprises:
(a) a DNA sequence as depicted in any of Seq. Ids 11, 13, 15, 17 or 19;
(b) a DNA sequence encoding a polypeptide comprising the primary structural conformation of amino acid numbers 401 to 494 of tTG or the primary structural conformation of amino acid numbers 401 to 491 of tTG, or one or more active fragments of:
amino acid numbers 401 to 494 of tTG or amino acid numbers 401 to 491 of tTG, with which autoantibodies produced in response to tTG can interact, under conditions that allow interaction of tTG with such autoantibodies,
with the exception of human, guinea pig, mouse, bovine or rat full length tTG;
(c) a DNA sequence encoding a polypeptide comprising the primary structural conformation of amino acids as depicted in one or more of Seq. Ids 12, 14, 16, 18 and 20, or one or more active fragments of amino acids as depicted in one or more of FIGS. 18, 20, 22, 24 and 26, with which autoantibodies produced in response to tTG can interact, under conditions that allow interaction of tTG with such autoantibodies, with the exception of human, guinea pig, mouse, bovine or rat full length tTG;
(d) a DNA sequence differing from the DNA sequence of (a) in codon sequence due to the degeneracy of the genetic code;
(e) a DNA sequence comprising a fragment or an allelic variation of the DNA sequence of (a); and
(f) a DNA sequence which hybridizes to any of DNA sequences (a), (b), (c), (d) or (e) and encodes a polypeptide with which autoantibodies produced in response to tTG can interact, under conditions that allow interaction of tTG with such autoantibodies, and which comprises part or all of the primary structural conformation of one or more epitopes of tTG with which autoantibodies produced in response to tTG can interact, under conditions that allow interaction of tTG with such autoantibodies.

7. A DNA sequence as depicted in any of Seq. Ids 1, 3, 5, 7, 9, 11, 13, 15, 17 or 19, or a DNA sequence differing therefrom due to the degeneracy of the genetic code.

8. A biologically functional plasmid or DNA vector including a DNA sequence as defined in claim 1.

9. A host cell which is transformed or transfected with a DNA sequence as defined in claim 1.

10. A polypeptide with which autoantibodies and/or lymphocytes produced in response to tTG can interact, under conditions that allow interaction of tTG with such autoantibodies or lymphocytes, and which comprises part or all of the primary structural conformation of one or more epitopes of tTG with which autoantibodies and/or lymphocytes produced in response to tTG can interact, under conditions that allow interaction of tTG with such autoantibodies or lymphocytes, which polypeptide comprises the primary structural conformation of amino acid numbers 1 to 89 of tTG, and/or the primary structural conformation of amino acid numbers 401 to 494 of tTG or the primary structural conformation of amino acid numbers 401 to 491 of tTG, or one or more active fragments of:
amino acid numbers 1 to 89 of tTG, and/or amino acid numbers 401 to 494 of tTG or amino acid numbers 401 to 491 of tTG, with which autoantibodies and/or lymphocytes produced in response to tTG can interact, under conditions that allow interaction of tTG with such autoantibodies or lymphocytes,
with the exception of human, guinea pig, mouse, bovine or rat full length tTG.

11. A polypeptide with which autoantibodies and/or lymphocytes produced in response to tTG can interact, under conditions that allow interaction of tTG with such autoantibodies or lymphocytes, and which comprises part or all of the primary structural conformation of one or more epitopes of tTG with which autoantibodies and/or lymphocytes produced in response to tTG can interact, under conditions that allow interaction of tTG with such autoantibodies or lymphocytes, which polypeptide comprises the primary structural conformation of amino acid numbers 1 to 89 of tTG, or one or more active fragments of amino acid numbers 1 to 89 of tTG, with which autoantibodies and/or lymphocytes produced in response to tTG can interact, under conditions that allow interaction of tTG with such autoantibodies or lymphocytes, with the exception of human, guinea pig, mouse, bovine or rat full length tTG.

12. A polypeptide with which autoantibodies and/or lymphocytes produced in response to tTG can interact, under conditions that allow interaction of tTG with such autoantibodies or lymphocytes, and which comprises part or all of the primary structural conformation of one or more epitopes of tTG with which autoantibodies and/or lymphocytes produced in response to tTG can interact, under conditions that allow interaction of tTG with such autoantibodies or lymphocytes, which polypeptide comprises the primary structural conformation of amino acid numbers 401 to 494 of tTG or the primary structural conformation of amino acid numbers 401 to 491 of tTG, or one or more active fragments of:
amino acid numbers 401 to 494 of tTG or amino acid numbers 401 to 491 of tTG, with which autoantibodies and/or lymphocytes produced in response to tTG can interact, under conditions that allow interaction of tTG with such autoantibodies or lymphocytes;
with the exception of human, guinea pig, mouse, bovine or rat full length tTG.

13. A polypeptide with which autoantibodies produced in response to tTG can interact, under conditions that allow interaction of tTG with such autoantibodies, and which comprises part or all of the primary structural conformation of one or more epitopes of tTG with which autoantibodies produced in response to tTG can interact, under conditions that allow interaction of tTG with such autoantibodies, which polypeptide comprises the primary structural conformation of amino acid numbers 1 to 89 of tTG, and/or the primary structural conformation of amino acid numbers 401 to 494 of tTG or the primary structural conformation of amino acid numbers 401 to 491 of tTG, or one or more active fragments of:
amino acid numbers 1 to 89 of tTG, and/or amino acid numbers 401 to 494 of tTG or amino acid numbers 401 to 491 of tTG, with which autoantibodies produced in response to tTG can interact, under conditions that allow interaction of tTG with such autoantibodies;
with the exception of human, guinea pig, mouse, bovine or rat full length tTG.

14. A polypeptide with which autoantibodies produced in response to tTG can interact, under conditions that allow interaction of tTG with such autoantibodies, and which comprises part or all of the primary structural conformation of one or more epitopes of tTG with which autoantibodies produced in response to tTG can interact, under conditions that allow interaction of tTG with such autoantibodies, which polypeptide comprises the primary structural conformation of amino acid numbers 1 to 89 of tTG, or one or more active fragments of amino acid numbers 1 to 89 of tTG, with which autoantibodies produced in response to tTG can interact, under conditions that allow interaction of tTG with such autoantibodies, with the exception of human, guinea pig, mouse, bovine or rat full length tTG.

15. A polypeptide with which autoantibodies produced in response to tTG can interact, under conditions that allow interaction of tTG with such autoantibodies, and which comprises part or all of the primary structural conformation of one or more epitopes of tTG with which autoantibodies produced in response to tTG can interact, under conditions that allow interaction of tTG with such autoantibodies, which polypeptide comprises the primary structural conformation of amino acid numbers 401 to 494 of tTG or the primary structural conformation of amino acid numbers 401 to 491 of tTG, or one or more active fragments of:
amino acid numbers 401 to 494 of tTG or amino acid numbers 401 to 491 of tTG, with which autoantibodies produced in response to tTG can interact, under conditions that allow interaction of tTG with such autoantibodies;
with the exception of human, guinea pig, mouse, bovine or rat full length tTG.

16. A polypeptide with which autoantibodies and/or lymphocytes produced in response to tTG can interact,under conditions that allow interaction of tTG with such autoantibodies or lymphocytes, and which comprises part or all of the primary structural conformation of one or more epitopes of tTG with which autoantibodies and/or lymphocytes produced in response to tTG can interact, under conditions that allow interaction of tTG with such autoantibodies or lymphocytes, which polypeptide comprises the primary structural conformation of amino acids as depicted in one or more of Seq. Ids 2, 4, 6, 8, 10, 12, 14, 16, 18 and 20, or one or more active fragments of amino acids as depicted in one or more of Seq. Ids 2, 4, 6, 8, 10, 12, 14, 16, 18 and 20, with which autoantibodies and/or lymphocytes produced in response to tTG can interact, under conditions that allow interaction of tTG with such autoantibodies or lymphocytes, with the exception of human, guinea pig, mouse, bovine or rat full length tTG.

17. A polypeptide with which autoantibodies and/or lymphocytes produced in response to tTG can interact, under conditions that allow interaction of tTG with such autoantibodies or lymphocytes, and which comprises part or all of the primary structural conformation of one or more epitopes of tTG with which autoantibodies and/or lymphocytes produced in response to tTG can interact, under conditions that allow interaction of tTG with such autoantibodies or lymphocytes, which polypeptide comprises the primary structural conformation of amino acids as depicted in one or more of Seq. Ids 2, 4, 6, 8 and 10, or one or more active fragments of amino acids as depicted in one or more of Seq. Ids 2, 4, 6, 8 and 10, with which autoantibodies and/or lymphocytes produced in response to tTG can interact, under conditions that allow interaction of tTG with such autoantibodies or lymphocytes, with the exception of human, guinea pig, mouse, bovine or rat full length tTG.

18. A polypeptide with which autoantibodies and/or lymphocytes produced in response to tTG can interact, under conditions that allow interaction of tTG with such autoantibodies or lymphocytes, and which comprises part or all of the primary structural conformation of one or more epitopes of tTG with which autoantibodies and/or lymphocytes produced in response to tTG can interact, under conditions that allow interaction of tTG with such autoantibodies or lymphocytes, which polypeptide comprises the primary structural conformation of amino acids as depicted in one or more of Seq. Ids 12, 14, 16, 18 and 20, or one or more active fragments of amino acids as depicted in one or more of Seq. Ids 12, 14, 16, 18 and 20, with which autoantibodies and/or lymphocytes produced in response to tTG can interact, under conditions that allow interaction of tTG with such autoantibodies or lymphocytes, with the exception of human, guinea pig, mouse, bovine or rat full length tTG.

19. A polypeptide with which autoantibodies produced in response to tTG can interact, under conditions that allow interaction of tTG with such autoantibodies, and which comprises part or all of the primary structural conformation of one or more epitopes of tTG with which autoantibodies produced in response to tTG can interact, under conditions that allow interaction of tTG with such autoantibodies, which polypeptide comprises the primary structural conformation of amino acids as depicted in one or more of Seq. Ids 2, 4, 6, 8, 10, 12, 14, 16, 18 and 20, or one or more active fragments of amino acids as depicted in one or more of Seq. Ids 2, 4, 6, 8, 10, 12, 14, 16, 18 and 20, with which autoantibodies produced in response to tTG can interact, under conditions that allow interaction of tTG with such autoantibodies, with the exception of human, guinea pig, mouse, bovine or rat full length tTG.

20. A polypeptide with which autoantibodies produced in response to tTG can interact, under conditions that allow interaction of tTG with such autoantibodies, and which comprises part or all of the primary structural conformation of one or more epitopes of tTG with which autoantibodies produced in response to tTG can interact, under conditions that allow interaction of tTG with such autoantibodies, which polypeptide comprises the primary structural conformation of amino acids as depicted in one or more of Seq. Ids 2, 4, 6, 8 and 10, or one or more active fragments of amino acids as depicted in one or more of Seq. Ids 2, 4, 6, 8 and 10, with which autoantibodies produced in response to tTG can interact, under conditions that allow interaction of tTG with such autoantibodies, with the exception of human, guinea pig, mouse, bovine or rat full length tTG.

21. A polypeptide with which autoantibodies produced in response to tTG can interact, under conditions that allow interaction of tTG with such autoantibodies, and which comprises part or all of the primary structural conformation of one or more epitopes of tTG with which autoantibodies produced in response to tTG can interact, under conditions that allow interaction of tTG with such autoantibodies, which polypeptide comprises the primary structural conformation of amino acids as depicted in one or more of Seq. Ids 12, 14, 16, 18 and 20, or one or more active fragments of amino acids as depicted in one or more of Seq. Ids 12, 14, 16, 18 and 20, with which autoantibodies produced in response to tTG can interact, under conditions that allow interaction of tTG with such autoantibodies, with the exception of human, guinea pig, mouse, bovine or rat full length tTG.

22. A polypeptide as depicted in any of Seq. Ids 2, 4, 6, 8, 10, 12, 14, 16, 18 and 20.

23. A process of preparing a polypeptide as defined in claim 10, which process comprises:
(i) providing a host cell which is transformed or transfected with a DNA sequence or a biologically functional plasmid or DNA vector;
(ii) growing the host cell; and
(iii) recovering a polypeptide as defined in claim 10.

24. A method of screening for autoantibodies or lymphocytes produced in response to tTG in a sample of body fluid obtained from a subject suspected of suffering from, susceptible to, having or recovering from autoimmune disease associated with an immune reaction to tTG, said method comprising:
(a) providing either (i) said sample of body fluid from said subject or (ii) lymphocytes isolated from said sample;
(b) contacting said sample or isolated lymphocytes with a polypeptide as defined in claim 10, under conditions that allow interaction of tTG with autoantibodies or lymphocytes produced in response to tTG, so as to permit said polypeptide to interact with autoantibodies, or lymphocytes, produced in response to tTG, and present in, or isolated from, said sample; and
(c) monitoring the degree, or effect, of interaction of said polypeptide with either said autoantibodies, or said lymphocytes, produced in response to tTG and present in, or isolated from, said sample, thereby providing an indication of the presence of said autoantibodies, or said lymphocytes, in said sample, or isolated from said sample.

25. A method according to claim 24, for screening for autoantibodies produced in response to tTG and present in said sample.

26. A method of screening for autoantibodies produced in response to tTG in a sample of body fluid obtained from a subject suspected of suffering from, susceptible to, having or recovering from autoimmune disease associated with an immune reaction to tTG, said method comprising:
(a) providing said sample of body fluid from said subject;
(b) contacting said sample with a polypeptide as defined in claim 10, under conditions that allow interaction of tTG with autoantibodies produced in response to tTG, so as to permit said polypeptide to interact with autoantibodies produced in response to tTG and present in said sample; and
(c) monitoring the degree of interaction of said polypeptide with said autoantibodies produced in response to tTG and present in said sample, thereby providing an indication of the presence of said autoantibodies in said sample.

27. A method according to claim 24, which comprises directly monitoring interaction of (i) autoantibodies to tTG present in the sample of body fluid from the subject and (ii) a polypeptide as defined in any of claims 10 to 22.

28. A method according to claim 24, which further comprises providing at least one competitor, whereby in step (b) a polypeptide as defined in any of claims 10 to 22, can interact with either the competitor or autoantibodies to tTG and present in said sample.

29. A method of screening for autoantibodies to tTG in a sample of body fluid obtained from a subject suspected of suffering from, susceptible to, having or recovering from autoimmune disease associated with an immune reaction to tTG, said method comprising:
(a) providing said sample of body fluid from said subject;
(b) contacting said sample with
(i) full length tTG, and
(ii) at least one competitor capable of competing with autoantibodies to tTG in the interaction thereof with a polypeptide as defined in claim 10,
under conditions that allow interaction of tTG with autoantibodies to tTG, so as to permit said full length tTG to interact with either autoantibodies to tTG present in said sample, or said competitor; and
(c) monitoring the interaction of said full length tTG with said autoantibodies present in said sample, thereby providing an indication of the presence of said autoantibodies to tTG in said sample.

30. A kit for screening for autoantibodies or lymphocytes produced in response to tTG in a sample of body fluid obtained from a subject suspected of suffering from, susceptible to, having or recovering from autoimmune disease associated with an immune reaction to tTG, said kit comprising:
(a) a polypeptide as defined in claim 10;
(b) means for contacting either (i) a sample of body fluid obtained from said subject, or (ii) lymphocytes isolated from a sample of body fluid obtained from said subject, with said polypeptide as defined in claims 10, under conditions that allow interaction of tTG with autoantibodies or lymphocytes produced in response to tTG, so as to permit said polypeptide to interact with autoantibodies, or lymphocytes, produced in response to tTG, and present in, or isolated from, said sample; and
(c) means for monitoring the degree, or effect, of interaction of said polypeptide with either said autoantibodies, or said lymphocytes, produced in response to tTG and present in, or isolated from, said sample, thereby providing an indication of the presence of said autoantibodies, or lymphocytes, in said sample, or isolated from said sample.

31. A kit according to claim 30, for screening for autoantibodies produced in response to tTG in said sample of body fluid.

32. A kit for screening for autoantibodies produced in response to tTG in a sample of body fluid obtained from a subject suspected of suffering from, susceptible to, having or recovering from autoimmune disease associated with an immune reaction to tTG, said kit comprising:
(a) a polypeptide as defined in claim 10;
(b) means for contacting a sample of body fluid obtained from said subject with said polypeptide as defined in claim 10, under conditions that allow interaction of tTG with autoantibodies produced in response to tTG, so as to permit said polypeptide to interact with autoantibodies produced in response to tTG and present in said sample; and
(c) means for monitoring the degree of interaction of said polypeptide with said autoantibodies produced in response to tTG and present in said sample, thereby providing an indication of the presence of said autoantibodies in said sample.

33. A kit according to claim 30, which comprises means for directly monitoring interaction of (i) autoantibodies to tTG present in the sample of body fluid from the subject and (ii) a polypeptide with which autoantibodies and/or lymphocytes produced in response to tTG can interact, under conditions that allow interaction of tTG with such autoantibodies or lymphocytes, and which comprises part or all of the primary structural conformation of one or more epitopes of tTG with which autoantibodies and/or lymphocytes produced in response to tTG can interact, under conditions that allow interaction of tTG with such autoantibodies or lymphocytes, which polypeptide comprises the primary structural conformation of amino acid numbers 1 to 89 of tTG, and/or the primary structural conformation of amino acid numbers 401 to 494 of tTG or the primary structural conformation of amino acid numbers 401 to 491 of tTG, or one or more active fragments of:
amino acid numbers 1 to 89 of tTG, and/or amino acid numbers 401 to 494 of tTG or amino acid numbers 401 to 491 of tTG, with which autoantibodies and/or lymphocytes produced in response to tTG can interact, under conditions that allow interaction of tTG with such autoantibodies or lymphocytes,
with the exception of human, guinea pig, mouse, bovine or rat full length tTG.

34. A kit according to of claim 30 to 32, which further comprises at least one competitor, whereby a polypeptide with which autoantibodies and/or lymphocytes produced in response to tTG can interact, under conditions that allow interaction of tTG with such autoantibodies or lymphocytes, and which comprises part or all of the primary structural conformation of one or more epitopes of tTG with which autoantibodies and/or lymphocytes produced in response to tTG can interact, under conditions that allow interaction of tTG with such autoantibodies or lymphocytes, which polypeptide comprises the primary structural conformation of amino acid numbers 1 to 89 of tTG, and/or the primary structural conformation of amino acid numbers 401 to 494 of tTG or the primary structural conformation of amino acid numbers 401 to 491 of tTG, or one or more active fragments of:
amino acid numbers 1 to 89 of tTG, and/or amino acid numbers 401 to 494 of tTG or amino acid numbers 401 to 491 of tTG, with which autoantibodies and/or lymphocytes produced in response to tTG can interact, under conditions that allow interaction of tTG with such autoantibodies or lymphocytes,
with the exception of human, guinea pig, mouse, bovine or rat full length tTG, can interact with either the competitor or autoantibodies to tTG present in said sample of body fluid being screened.

35. A kit for screening for autoantibodies to tTG in a sample of body fluid obtained from a subject suspected of suffering from, susceptible to, having or recovering from autoimmune disease associated with an immune reaction to tTG, said kit comprising:
(a) full length tTG;
(b) at least one competitor capable of competing with autoantibodies to tTG in the interaction thereof with a polypeptide as defined in claim 10;
(c) means for contacting said sample of body fluid from said subject, said full length tTG and said competitor, under conditions that allow interaction of tTG with autoantibodies to tTG, so as to permit said full length tTG to interact with either autoantibodies to tTG present in said sample, or said competitor; and
(d) means for monitoring the interaction of said full length tTG with said autoantibodies present in said sample, thereby providing an indication of the presence of said autoantibodies to tTG in said sample.

36. An antibody produced in response to one or more epitope regions of tTG, which epitope region comprises part or all of the primary structural conformation of amino acids 1 to 89 of tTG, or the primary structural conformation of amino acids 401 to 494 or 401 to 491 of tTG, or one or more fragments of an antibody produced in response to one or more epitope regions of tTG, which epitope region comprises part or all of the primary structural conformation of amino acids 1 to 89 of tTG, or the primary structural conformation of amino acids 401 to 494 or 401 to 491 of tTG.

37. An antibody according to claim 36, which is obtained by the Examples.

38. An antibody according to claim 36, which is monoclonal, recombinant or polyclonal.

39. An antibody according to claim 38, which is monoclonal.

40. A host cell containing an antibody as defined in claims 36.

41. A hybridoma capable of secreting a monoclonal antibody as defined in claim 36.

42. For use in diagnosis, an antibody as defined in claim 36.

43. Use according to claim 42, in the diagnosis of autoimmune disease associated with an immune reaction to tTG.

44. For use in therapy, an antibody as defined in claim 36.

45. Use according to claim 44, in the therapeutic treatment of autoimmune disease associated with an immune reaction to tTG.

46. Use according to claim 43, wherein the autoimmune disease is coeliac disease.

47. An antibody according to claim 36, in the manufacture of a medicament for the treatment of coeliac disease.

48. For use in diagnosis, a polypeptide as defined in claim 10.

49. Use according to claim 48, in the diagnosis of autoimmune disease associated with an immune reaction to tTG.

50. For use in therapy, a polypeptide as defined in claim 10.

51. Use according to claim 50, in the therapeutic treatment of autoimmune disease associated with an immune reaction to tTG.

52. Use according to claim 49, wherein the autoimmune disease is coeliac disease.

53. A pharmaceutical composition comprising a polypeptide as defined in claim 10, together with a pharmaceutically acceptable carrier, diluent or excipient therefor, wherein said polypeptide can interact with autoantibodies and/or lymphocytes produced in response to tTG.

54. A polypeptide as defined in claim 10, for use in the manufacture of a medicament for the treatment of coeliac disease.

55. A method of diagnosing the likely onset or presence of autoimmune disease associated with an immune reaction to tTG in a subject suspected of suffering from, susceptible to, having or recovering from, autoimmune disease associated with an immune reaction to tTG, the method comprising detecting autoantibodies or lymphocytes produced in response to tTG in a sample of body fluid from the subject according to a method as defined in claim 24, and whereby the detected autoantibodies and/or lymphocytes can provide a diagnosis of the likely onset or presence of autoimmune disease associated with an immune reaction to tTG in the subject

56. A method of delaying or preventing the onset of autoimmune disease associated with an immune reaction to tTG in an animal subject suspected of suffering from, susceptible to or recovering from autoimmune disease associated with an immune reaction to tTG, which method comprises initially detecting autoantibodies or lymphocytes indicative of the onset or presence of autoimmune disease associated with an immune reaction to tTG in a sample of body fluid obtained from the subject according to a method as defined in claim 24, thereby providing a diagnosis of the likely onset of autoimmune disease associated with an immune reaction to tTG in the subject, and thereafter therapeutically treating the subject so as to delay the onset and/or prevent autoimmune disease associated with an immune reaction to tTG.

57. A method of monitoring dietary compliance by a subject having, suspected of suffering from, susceptible to or recovering from gluten sensitive enteropathy, which method comprises screening for autoantibodies or lymphocytes produced in response to tTG in a sample of body fluid obtained from the subject according to a method as defined in claim 24, and whereby the detection of such autoantibodies and/or lymphocytes produced in response to tTG in the sample of body fluid provides an indication as to the dietary compliance of the subject to a gluten-free or substantially gluten free diet.

58. A method of treating autoimmune disease associated with an immune reaction to tTG in a subject, which method comprises initially detecting autoantibodies or lymphocytes produced in response to tTG in a sample of body fluid obtained from the subject according to a method as defined in claim 24, thereby providing a diagnosis of autoimmune disease in the subject, and administering to the subject a therapeutically effective amount of at least one therapeutic agent effective in the treatment of such autoimmune disease.

59. A method of treating autoimmune disease associated with an immune reaction to tTG in a subject, which method comprises administering to the subject a therapeutically effective amount of a therapeutic agent identified as providing a therapeutic effect by interaction with amino acids 1 to 89 of tTG, and/or amino acids 401 to 494 of tTG, or amino acids 401 to 492 of tTG.

60. A therapeutic agent identified as providing a therapeutic effect by interaction with amino acids 1 to 89 of tTG, and/or amino acids 401 to 494 of tTG, or amino acids 401 to 491 of tTG.

61. A therapeutic agent according to claim 60, for use in the therapeutic treatment of an autoimmune disease associated with an immune reaction to tTG.

62. A method of cloning lymphocytes produced in response to tTG, which method comprises:
providing a source of lymphocytes;
contacting the lymphocytes with a polypeptide as defined in claim 10, so as to effect proliferation of said lymphocytes; and
isolating and cloning the proliferating lymphocytes.

63. Use of lymphocytes prepared according to claim 62, in the therapeutic treatment of autoimmune disease associated with an immune reaction to tTG.

64. A pharmaceutical composition comprising lymphocytes prepared according to claim 62, together with a pharmaceutically acceptable carrier, diluent or excipient therefor.

65. Use of lymphocytes prepared according to claim 62, in the manufacture of a medicament for the treatment of autoimmune disease associated with an immune reaction to Ttg.

66. Use of lymphocytes prepared according to claim 62, in the manufacture of a medicament for the treatment of coeliac disease.

67. A biologically functional plasmid or DNA vector including a DNA sequence as defined in any of claim 2.

68. A biologically functional plasmid or DNA vector including a DNA sequence as defined in any of claim 3

69. A biologically functional plasmid or DNA vector including a DNA sequence as defined in any of claim 4.

70. A biologically functional plasmid or DNA vector including a DNA sequence as defined in any of claim 5.

71. A biologically functional plasmid or DNA vector including a DNA sequence as defined in any of claim 6.

72. A biologically functional plasmid or DNA vector including a DNA sequence as defined in any of claim 7.

73. A host cell which is transformed or transfected with a plasmid or vector as defined in claim 67.

74. A host cell which is transformed or transfected with a plasmid or vector as defined in claim 68.

75. A host cell which is transformed or transfected with a plasmid or vector as defined in claim 69.

76. A host cell which is transformed or transfected with a plasmid or vector as defined in claim 70.

77. A host cell which is transformed or transfected with a plasmid or vector as defined in claim 71.

78. A host cell which is transformed or transfected with a plasmid or vector as defined in claim 72.
